(19) United States
(12) Patent Application Publication
(10) Pub. No.: US 2015/0183818 A1
(43) Pub. Date: Jul. 2, 2015

(54) PROCESS FOR PREPARING DIASTEREOMERICALLY ENRICHED PHOSPHORAMIDATE DERIVATIVES OF NUCLEOSIDE COMPOUNDS FOR TREATMENT OF VIRAL INFECTIONS

(71) Applicant: BRISTOL-MYERS SQUIBB COMPANY, Princeton, NJ (US)

(72) Inventors: Kristy Tran, Manalapan, NJ (US); Martin D. Eastgate, Titusville, NJ (US); Jacob Janey, New York, NY (US); Ke Chen, East Brunswick, NJ (US); Victor W. Rosso, Monroe, NJ (US)

(73) Assignee: BRISTOL-MYERS SQUIBB COMPANY, Princeton, NJ (US)

(21) Appl. No.: 14/409,523

(22) PCT Filed: Jul. 2, 2013
(86) PCT No.: PCT/US13/49031

§ 371 Date: Dec. 19, 2014

Related U.S. Application Data
(60) Provisional application No. 61/667,620, filed on Jul. 3, 2012.

(51) Int. Cl. C07H 19/20; C07F 9/24; C07H 19/10

(52) U.S. Cl. CPC C07H 19/20; C07H 19/10; C07F 9/2458

(57) ABSTRACT

The present invention is directed to a process for preparing diastereomerically enriched nucleoside phosphoramidates having the formula I:

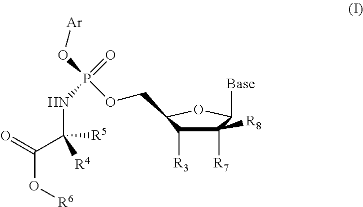

### RELATED APPLICATIONS

This application claims benefit of U.S. Provisional Application No. 61/667,620, filed on Jul. 3, 2012, which is herein incorporated by reference in its entirety.

### FIELD OF THE INVENTION

This application relates to novel methods for preparing nucleoside phosphoramidates that are useful as agents for treating viral diseases.

### BACKGROUND OF THE INVENTION

HCV is a member of the Flaviviridae family of RNA viruses that affect animals and humans. The genome is a single 9.6-kilobase strand of RNA, and consists of one open reading frame that encodes for a polyprotein of approximately 3000 amino acids flanked by untranslated regions at both 5′ and 3′ ends (5′- and 3′-UTR). The polyprotein serves as the precursor to at least 10 separate viral proteins critical for replication and assembly of progeny viral particles.

Hepatitis C Virus (HCV) infection is a major health problem that leads to chronic liver disease, such as cirrhosis and hepatocellular carcinoma, in a substantial number of infected individuals, estimated to be 2-15% of the world population. There are an estimated 4.5 million infected people in the United States alone, according to the U.S. Center for Disease control. According to the World Health Organization, there are more than 200 million infected individuals worldwide, with at least 3 to 4 million people being infected each year. Once infected, about 20% of people clear the virus, but the remainder can harbor HCV for the rest of their lives.

Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring.

At present, the standard treatment for chronic HCV is interferon alpha (IFN-alpha) in combination with ribavirin, which requires at least six (6) months of treatment. However, treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction.

International patent publication WO 2010/081082 discloses novel phosphoramidate nucleoside prodrugs with improved properties over known therapeutics. These compounds exist as diastereomeric or enantiomeric mixtures, potentially complicating development of these compounds into pharmaceutically acceptable compounds, which results in increased manufacturing costs and potential limitations to access. There is a need for an efficient and selective process for the preparation of diastereomerically enriched nucleoside phosphoramidates. WO 2008/121634, WO 2011/123668 and WO 2012/012465 disclose processes for preparing nucleoside phosphoramidate prodrugs that result in diastereomerically enriched product. These methods rely on a very well precedented process—a nucleophilic substitution at phosphorous which is well known to proceed with inversion of stereochemistry at phosphorous. The inversion of stereochemistry at phosphorous during a nucleophilic substitution has been documented as early as 1962 (Green, M. et al., Proc. Chem. Soc., 307 (1962); Angew. Chem. Int. Ed., 2:11 (1963)). In order to form diastereomerically enriched phosphoramidate drug compounds, previous work has involved the isolation of an activated phosphate containing a leaving group (generally —OC₆F₅ or —OC₆H₄-pNO₂), which would then be coupled to the nucleoside to yield the desired compound. In order to obtain diastereomeric enrichment, these activated phosphates are isolated separately and re-crystallized to diastereomeric purity, often in exceptionally low yields. Thus, the stereochemical information is introduced through crystallization. These approaches are therefore extremely limited, only compounds where a fractional crystallization is possible can be employed, exhibit poor overall yields, and are cumbersome to perform.

### SUMMARY OF THE INVENTION

The present invention leverages a highly diastereoselective coupling process to convert a pro-chiral phosphorous atom into a diastereomerically enriched phosphate through a selective reaction. This new process offers many significant advantages in terms of yield, ease of operation and cost. In some aspects of the present invention, methods for preparing a compound of formula Ia having the following structure, or a pharmaceutically acceptable salt thereof:

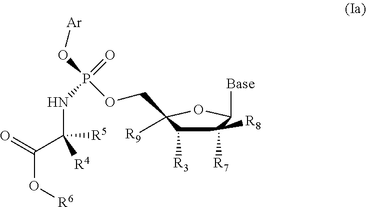

wherein

Base is a naturally occurring or modified purine or pyrimidine base linked to the furanose ring through a carbon or nitrogen atom;

Ar is selected from phenyl, naphthyl,

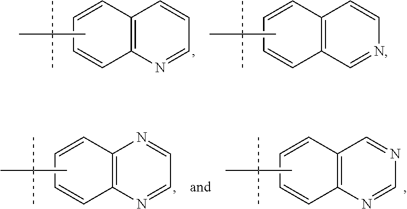

any of which are optionally substituted with C₁-C₆alkyl, C₁-C₆alkoxy, di(C₁-C₆)alkylamino or C₁-C₆alkylcarboxy(C₁-C₆)alkyl-;

R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;

R⁴ and R⁵ are independently selected from hydrogen, C₁-C₆alkyl optionally substituted with alkylthio, benzyl optionally substituted with one or more halo, C₁-C₆alkyl, or C₁-C₆alkoxy, phenyl optionally substituted with one or more halo, C₁-C₆alkyl, or C₁-C₆alkoxy;

R⁶ is selected from C₁-C₁₀alkyl, C₃-C₈cycloalkyl, C₃-C₈cycloalkyl-alkyl-, phenyl(C₁-C₆)alkyl- optionally substituted with C₁-C₆alkyl, C₁-C₆alkoxy, and halo, indanyl and heterocycloalkyl;

R⁷ is selected from the group consisting of H, alkyl, —OH, OP, wherein P is a protecting group, OCH₃, halo, NH₂;

R⁸ is selected from the group consisting of H, CH₃, CH₂F, CHF₂, CF₃, F, CN, —OH, —OP wherein P is a protecting group, halo, alkyl, alkenyl, and alkynyl; and

R⁹ is selected from the group consisting of H, C₁₋₄ alkyl, CN, halo, —OH, —CH₂CN, —CH₂NH₂, vinyl, C₂-C₄ alkynyl, O—C₁₋₆ alkyl, —CH₂F, N₃, in the presence of an activator, a base, and optionally an additive;

comprising contacting a compound having the following Formula II, or a salt thereof:

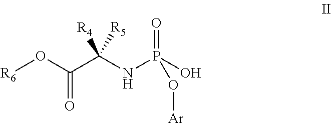

with a nucleoside compound of Formula IIIa:

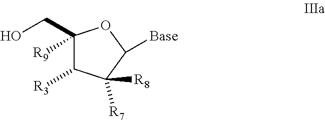

wherein

R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;

R⁷ is selected from the group consisting of H, alkyl, —OH, OP, wherein P is a protecting group, OCH₃, halo, N₃, NH₂;

R⁸ is selected from the group consisting of H, CH₃, CH₂F, CHF₂, CF₃, F, CN, —OH, —OP wherein P is a protecting group, halo, alkyl, alkenyl, and alkynyl; and

R⁹ is selected from the group consisting of H, C₁₋₄ alkyl, CN, halo, —OH, —CH₂CN, —CH₂NH₂, vinyl, C₂-C₄ alkynyl, —O—C₁₋₆ alkyl, and —CH₂F, N₃,

in the presence of an activator, a base, and optionally an additive.

According to some embodiments of the present invention methods for preparing the following nucleoside phosphoramidate compound of Formula I, or a pharmaceutically acceptable salt thereof are provided:

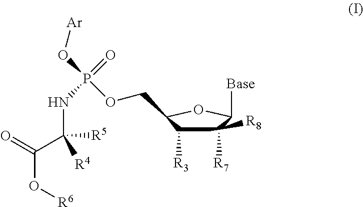

wherein

Base is a naturally occurring or modified purine or pyrimidine base linked to the furanose ring through a carbon or nitrogen atom;

Ar is selected from phenyl, naphthyl,

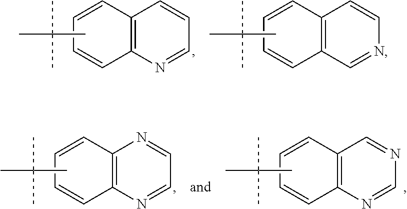

any of which are optionally substituted with C₁-C₆alkyl, C₁-C₆alkoxy, di(C₁-C₆)alkylamino or C₁-C₆alkylcarboxy(C₁-C₆)alkyl-;

R³ is O or —OH;

R⁴ and R⁵ are independently selected from hydrogen, C₁-C₆alkyl optionally substituted with alkylthio, benzyl optionally substituted with one or more halo, C₁-C₆alkyl, or C₁-C₆alkoxy, phenyl optionally substituted with one or more halo, C₁-C₆alkyl, or C₁-C₆alkoxy; and

R⁶ is selected from C₁-C₁₀alkyl, C₃-C₈cycloalkyl, C₃-C₈cycloalkyl-alkyl-, phenyl(C₁-C₆)alkyl- optionally substituted with C₁-C₆alkyl, C₁-C₆alkoxy, and halo, indanyl and heterocycloalkyl;

comprising contacting a compound having Formula II, or a salt thereof:

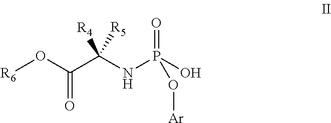

with a nucleoside compound of Formula III:

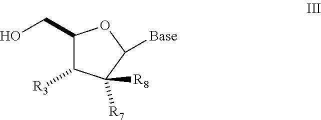

wherein

R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;

R⁷ is selected from the group consisting of-OH, halo, and alkyl; and

R⁸ is selected from the group consisting of —OH, halo, alkyl, alkenyl, and alkynyl; in the presence of an activator, a base, and optionally an additive.

### DETAILED DESCRIPTION OF THE INVENTION

### Definitions

As used herein, the term “alkyl” refers to a straight or branched saturated monovalent cyclic or acyclic hydrocarbon radical, having the number of carbon atoms as indicated (or where not indicated, an acyclic alkyl group preferably has 1-20, more preferably 1-6 (lower alkyl), more preferably 1-4 carbon atoms and a cyclic alkyl group preferably has 3-20, preferably 3-10, more preferably 3-7 carbon atoms), optionally substituted with one, two, three or more substituents independently selected from the group set out above. By way of non-limiting examples, suitable alkyl groups include methyl, ethyl, propyl, butyl, pentyl, hexyl, octyl, nonyl, isopropyl, 2-butyl, cyclopropyl, cyclohexyl, cyclopentyl, neopentyl and dodecyl. The term “C₃-C₈cycloalkyl” refers to cyclic alkyl group comprising from about 3 to about 8 C atoms. The term “C₃-C₈cycloalkyl-alkyl” refers to an acyclic alkyl group substituted by a cyclic alkyl group comprising from about 3 to about 8 C atoms.

As used herein, the term “alkenyl” refers to a straight or branched unsaturated monovalent acyclic or cyclic hydrocarbon radical having one or more C═C double bonds and having the number of carbon atoms as indicated (or where not indicated, an acyclic alkenyl group preferably has 2-20, more preferably 2-6, more preferably 2-4 carbon atoms and a cyclic alkenyl group preferably has 4-20, more preferably 4-6 carbon atoms), optionally substituted with one, two, three or more substituents independently selected from the group set out above. By way of non-limiting examples, suitable alkenyl groups include vinyl, propenyl, butenyl, pentenyl and hexenyl.

As used herein, the term “alkynyl” refers to a straight or branched unsaturated monovalent acyclic or cyclic hydrocarbon radical having one or more triple C/C bonds and having the number of carbon atoms as indicated (or where not indicated, an acyclic alkynyl group preferably has 2-20, more preferably 2-6, more preferably 2-4 carbon atoms and a cyclic alkynyl group preferably has 7-20, more preferably 8-20 carbon atoms), optionally substituted with one, two, three or more substituents independently selected from the group set out above.

As used herein, the term “alkoxy” or the term “alkyloxy” refers to the group alkyl-O—, where alkyl is as defined above and where the alkyl moiety may optionally be substituted by one, two, three or more substituents as set out above for alkyl. By way of non-limiting examples, suitable alkoxy groups include methoxy, ethoxy, n-propoxy, iso-propoxy, n-butoxy, tert-butoxy, sec-butoxy, n-pentoxy, n-hexoxy and 1,2-dimethylbutoxy. The term “cycloalkyloxy” refers to the group cyclicalkyl-O—, where cyclicalkyl is as defined above and where the cyclicalkyl moiety may be optionally substituted by one, two, three or more substituents as set out above for alkyl.

As used herein, the term “alkylthio” refers to the group alkyl-S—, where alkyl is as defined above and where the alkyl moiety may optionally be substituted by one, two, three or more substituents as set out above for alkyl. By way of non-limiting examples, suitable alkylthio groups include methylthio, ethylthio, n-propylthio, iso-propylthio, n-butylthio, tert-butylthio, sec-butylthio, n-pentylthio, n-hexoxy and 1,2-dimethylbutylthio.

The term “alkylamino” refers to a group alkyl-NR¹R²—, wherein R¹ and R² are H, alkyl, aryl and where alkyl is defined as above.

As used herein, the term “aryloxy” refers to the group aryl-O—, where aryl is as defined below and where the aryl moiety may optionally be substituted by one, two, three or more substituents as set out above with respect to the group Ar.

As used herein, the term “alkoxyalkyl” refers to an alkyl group having an alkoxy substituent. Binding is through the alkyl group. The alkyl moiety and the alkoxy moiety are as defined herein with respect to the definitions of alkyl and alkoxy, respectively. The alkoxy and alkyl moieties may each be substituted by one, two, three or more substituents as set out above with regard to the definition of alkyl.

As used herein, the term “alkylthioalkyl” refers to an alkyl group having an alkylthio substituent. Binding is through the alkyl group. The alkyl moiety and the alkylthio moiety are as defined herein with respect to the definitions of alkyl and alkylthio, respectively. The alkylthio and alkyl moieties may each be substituted by one, two, three or more substituents as set out above with regard to the definition of alkyl.

As used herein, the term “alkoxyaryl” refers to an aryl group having an alkoxy substituent. Binding is through the aryl group. The alkoxy moiety and the aryl moiety are as defined herein with respect to the definitions of alkoxy and aryl, respectively. The alkoxy and aryl moieties may each be substituted by one, two, three or more substituents, as defined herein with regard to the definitions of alkoxy and aryl, respectively.

As used herein, the term “cycloalkylaryl” refers to an aryl group having a cyclic alkyl substituent. Binding is through the aryl group. The cycloalkyl moiety and the aryl moiety are as defined herein with respect to the definitions of cycloalkyl and aryl, respectively.

As used herein, the term “aryl(C₁-C₆)alkyl-” refers to a C₁-C₆ alkyl group substituted at any carbon by an aryl group. Binding is through the alkyl group. The aryl moiety and the alkyl moiety are as defined herein with respect to the definitions of aryl and alkyl. The aryl group may be substituted. By way of non-limiting examples, suitable aryl(C₁-C₆)alkyl- groups include benzyl, 1-phenylethyl, 3-phenylpropyl, 4-chlorobenzyl, 4-fluorobenzyl, 2,4-difluorobenzyl, and the like.

As used herein, the term “alkylcarboxy(C₁-C₆)alkyl-” refers to a C₁-C₆ alkyl group substituted at any carbon by an alkyl carboxy [alkyl-C(═O)O—] group. The alkyl moiety is as defined hereinabove. By way of non-limiting examples, suitable alkylcarboxy(C₁-C₆)alkyl- groups include acetoxymethyl[CH₃C(═O)O—CH₂-], propanoyloxyethyl[CH₃CH₂C(═O)O—CH₂CH₂-], neo-pentoyloxypropyl [(CH₃)₃CCH₂C(═O)O—CH₂ CH₂CH₂-] and the like.

A cycloalkyl moiety and the aryl moiety may each be optionally substituted by one, two, three or more substituents as set out herein with regard to the definitions of alkyl and aryl, respectively.

As used herein the term “aryl” refers to a monovalent unsaturated aromatic carbocyclic radical having one, two, three, four, five or six rings, preferably one, two or three rings, which may be fused or bicyclic. An aryl group may optionally be substituted by one, two, three or more substituents as set out above with respect to optional substituents that may be present on the group Ar. Preferred aryl groups are: an aromatic monocyclic ring containing 6 carbon atoms; an aromatic bicyclic or fused ring system containing 7, 8, 9 or 10 carbon atoms; or an aromatic tricyclic ring system containing 10, 11, 12, 13 or 14 carbon atoms. Non-limiting examples of aryl include phenyl and naphthyl. These compounds may include substituent groups, preferably those substituent groups independently selected from hydroxy (—OH), acyl (R′—C(═O)), acyloxy (R′—C(O)—O—), nitro (—NO₂), amino (—NH₂), carboxyl (—COOH), cyano (—CN), C₁-C₆monoalkylamino, C₁-C₆dialkylamino, thiol, chloro, bromo, fluoro, iodo, SO₃H, —SH, —SR′, wherein R′ is independently selected from halo, C₁-C₆alkoxy, and C₁-C₆alkyl.

As used herein, the term “heterocycloalkyl” refers to a saturated or partially unsaturated heterocyclic ring system having one, two, three, four, five or six rings, preferably one, two or three rings, which may be fused or bicyclic, and having contained within the ring or rings at least one member selected from the group consisting of N, O and S. The prefix “C₅-C₂₀” or “C₅-C₁₀” used before heterocycloalkyl means, respectively, a five- to twenty- or a five- to ten-membered ring system at least one of which members is selected from the group consisting of N, O and S. Preferred heterocycloalkyl systems are: a monocyclic ring system having five members of which at least one member is a N, O or S atom and which optionally contains one additional O atom or one, two or three additional N atoms; a monocyclic ring having six members of which one, two or three members are a N or O atom; a bicyclic ring system having nine members of which at least one member is a N, O or S atom and which optionally contains one, two or three additional N atoms; or a bicyclic ring system having ten members of which one, two or three members are a N atom. By way of non-limiting examples, suitable heterocycloalkyl groups include tetrahydrofuranyl, tetrahydropyranyl, pyrrolidinyl, morpholinyl, tetrahydrothiopyranyl, thiomorpholinyl, and piperidinyl.

As used herein, the term “indanyl” refers to the fused bicyclic substituent of structure,

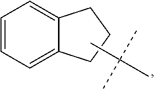

wherein the point of attachment of the radical to the rest of the molecule is on any available non-aromatic carbon atom.

Available carbon atoms and/or heteroatoms of the “heterocycloalkyl” ring systems described above may be substituted on the ring with one or more heteroatoms. Where the ring(s) is substituted with one or more heteroatoms, heteroatom substituents are selected from oxygen, nitrogen, sulphur and halogen (F, Cl, Br and I). Where the ring(s) is substituted with one or more heteroatoms, preferably there are 1, 2, 3 or 4 heteroatom substituents selected from the group consisting of oxygen, nitrogen and/or halogen. Preferred substituent groups are independently selected from hydroxy, acyl, acyloxy, nitro, amino, SO₃H, SH, SR′, wherein R′ is independently selected from the same groups as R; carboxyl, cyano, (C₁-C₆)alkylamino, (C₁-C₆)dialkylamino, thiol, chloro, bromo, fluoro and iodo.

The term “pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The “S” and “R” designations as used herein are determined using the Cahn-Ingold-Prelog method.

The term “diastereomerically enriched” as used herein refers to an instance where, due to the chirality at phosphorus, the mole amount of one diastereomer (Rp or Sp) exceeds the mole amount of the other diastereomer. Recognizing that the phosphorus atoms in the compounds of the present invention are chiral, one of ordinary skill will understand that a composition, comprised of the compounds of the present invention (e.g., a composition of compounds of Formula I, Ia, II, IV, VI, X, XII, respectively, comprise mixtures of diastereomers. “Diastereomerically enriched” means a composition having at least about 51 mol % to about 100 mol % of one diastereomer (Sp or Rp) and at most 49 mol % to 0 mol % of the other enantiomer (Rp or Sp). Within this meaning, “diastereomerically enriched” includes a composition comprised of about at least about 60 mol % of one diastereomer to about 40 mol % of the other, about 70 mol % of one diastereomer to about 30 mol % of the other, about 80 mol % of one diastereomer to about 20 mol % of the other, about 90 mol % of one diastereomer to about 10 mol % of the other, about 95 mol % of one diastereomer to about 5 mol % of the other, about 97 mol % to about 5 mol % of the other, about 98 mol % to about 2 mol % of the other, of about 99 mol % of diastereomer to about 1 mol % of the other, about 99.5 mol % of one diastereomer to about 0.5 mol % of the other, about 99.9 mol % of one diastereomer to about 0.1 mol % of the other.

The term “salt” refers to organic and inorganic salt forms of the phosphoric acids of Formula II. These salts may be derived from a variety of organic and inorganic counter ions well known in the art and include, by way of example, quinine salt, DBU salt, calcium salts and zinc salts. These salt forms may also contain a range of hydrates and solvates.

Unless otherwise indicated, the term “lower alkylthio”, “alkylthio”, “arylthio” or “aralkylthio” as employed herein alone or as part of another group includes any of the above alkyl, aralkyl or aryl groups linked to a sulfur atom.

The term “acyl” as used herein alone or as part of another group refers to a radical linked to a carbonyl (C═O) group which radical can be, for example, lower alkyl, aryl, heterocyclo, heteroaryl, cycloalkyl, lower alkoxy or amino

The term “naturally occurring or modified purine or pyrimidine base” refers to those naturally occurring and modified nucleoside bases such as adenine, N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-allylaminopurine, N⁶-thioallyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-5-iodopyrimidine, C⁶-iodo-pyrimidine, C⁵—Br-vinyl pyrimidine, C⁵—Br-vinyl-pyrimidine, C⁵-nitropyrimidine, C⁵-amino-pyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 2,6-diaminopurine, and 6-chloropurine. Additional non-classical purine bases include pyrrolo[1,2-f][1,2,4]triazines, imidazo[1,5-f][1,2,4]triazines, imidazo[1,2-f][1,2,4]triazines, and [1,2,4]triazolo[4,3-f][1,2,4]triazines, all of which are optionally substituted. The purine and pyrimidine bases of Formula I are linked to the ribose sugar, or analog thereof, through a nitrogen atom or carbon atom of the base. Functional oxygen and nitrogen groups on the base can be protected as necessary or desired. Suitable protecting groups are well known to those skilled in the art, and include, but are not limited to, trimethylsilyl, dimethylhexylsilyl, t-butyldimethylsilyl, and t-butyldiphenylsilyl, trityl, alkyl groups, and acyl groups such as acetyl and propionyl, methanesulfonyl, and p-toluenesulfonyl.

N₃ refers to azido or —N═N═NH.

The term “activator” as used herein refers to a reagent, reactant or combination of reagents capable of performing a formal dehydration of the phosphoric acid.

The term “additive” as used herein refers to a reagent, reactant or combination of reagents, which, when added to the reaction, either increase the rate or reaction, the overall yield of the reaction, or impacts the diastereoselectivity of the process.

The present invention is directed to novel processes for preparing diastereomerically enriched (at phosphorus) nucleoside phosphoramidate compounds having the following Formula Ia:

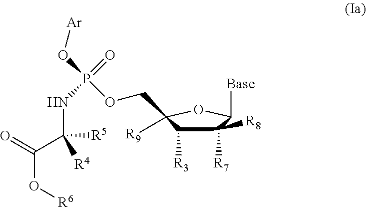

wherein

Base is a naturally occurring or modified purine or pyrimidine base linked to the furanose ring through a carbon or nitrogen atom;

Ar is selected from phenyl, naphthyl,

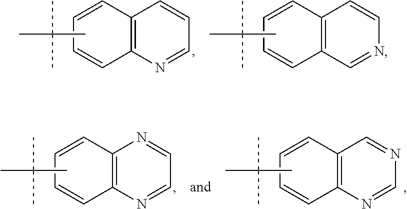

any of which are optionally substituted with C₁-C₆alkyl, C₁-C₆alkoxy, di(C₁-C₆)alkylamino or C₁-C₆alkylcarboxy(C₁-C₆)alkyl-;

R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;

R⁴ and R⁵ are independently selected from hydrogen, C₁-C₆alkyl optionally substituted with alkylthio, benzyl optionally substituted with one or more halo, C₁-C₆alkyl, or C₁-C₆alkoxy, phenyl optionally substituted with one or more halo, C₁-C₆alkyl, or C₁-C₆alkoxy;

R⁶ is selected from C₁-C₁₀alkyl, C₃-C₈cycloalkyl, C₃-C₈cycloalkyl-alkyl-, phenyl(C₁-C₆)alkyl- optionally substituted with C₁-C₆alkyl, C₁-C₆alkoxy, and halo, indanyl and heterocycloalkyl;

R⁷ is selected from the group consisting of H, alkyl, —OH, OP, wherein P is a protecting group, OCH₃, halo, NH₂;

R⁸ is selected from the group consisting of H, CH₃, CH₂F, CHF₂, CF₃, F, CN, —OH, —OP wherein P is a protecting group, halo, alkyl, alkenyl, and alkynyl; and

R⁹ is selected from the group consisting of H, C₁₋₄ alkyl, CN, halo, —OH, —CH₂CN, —CH₂NH₂, vinyl, C₂-C₄ alkynyl, O—C₁₋₆ alkyl, —CH₂F, N₃, in the presence of an activator, a base, and optionally an additive;

comprising contacting a compound having the following Formula II, or a salt thereof:

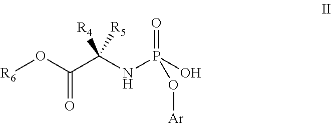

with a nucleoside compound of Formula IIIa:

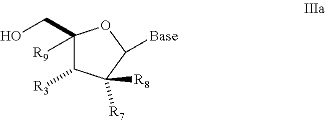

wherein

R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;

R⁷ is selected from the group consisting of H, alkyl, —OH, OP, wherein P is a protecting group, OCH₃, halo, N₃, NH₂;

R⁸ is selected from the group consisting of H, CH₃, CH₂F, CHF₂, CF₃, F, CN, —OH, —OP wherein P is a protecting group, halo, alkyl, alkenyl, and alkynyl; and

R⁹ is selected from the group consisting of H, C₁₋₄ alkyl, CN, halo, —OH, —CH₂CN, —CH₂NH₂, vinyl, C₂-C₄ alkynyl, —O—C₁₋₆ alkyl, and —CH₂F, N₃,

in the presence of an activator, a base, and optionally an additive.

According to some embodiments of the present invention, methods are provide for preparing diastereomerically enriched (at phosphorus) nucleoside phosphoramidate compounds having the following Formula I:

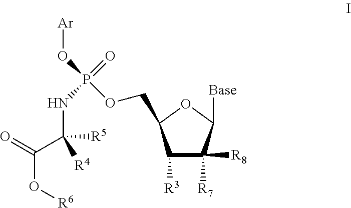

wherein

Base is a naturally occurring or modified purine or pyrimidine base linked to the furanose ring through a carbon or nitrogen atom;

Ar is selected from phenyl, naphthyl,

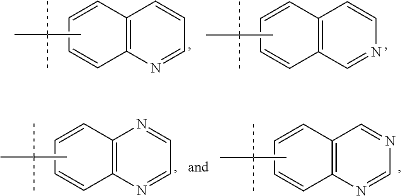

any of which are optionally substituted with C₁-C₆alkyl, C₁-C₆alkoxy, di(C₁-C₆)alkylamino or C₁-C₆alkylcarboxy(C₁-C₆)alkyl-;

R³ is O or —OH;

R⁴ and R⁵ are independently selected from hydrogen, C₁-C₆alkyl optionally substituted with alkylthio, benzyl optionally substituted with one or more halo, C₁-C₆alkyl, or C₁-C₆alkoxy, phenyl optionally substituted with one or more halo, C₁-C₆alkyl, or C₁-C₆alkoxy; and

R⁶ is selected from C₁-C₁₀alkyl, C₃-C₈cycloalkyl, C₃-C₈cycloalkyl-alkyl-, phenyl(C₁-C₆)alkyl- optionally substituted with C₁-C₆alkyl, C₁-C₆alkoxy, and halo, indanyl and heterocycloalkyl;

comprising contacting a compound having the following Formula II, or a salt thereof:

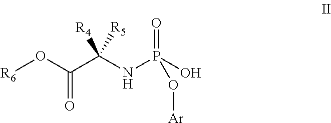

with a nucleoside compound of Formula III:

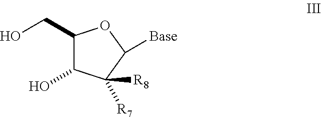

wherein

R⁷ is selected from the group consisting of —OH, —OP, wherein P is a protecting group, halo, and alkyl; and

R⁸ is selected from the group consisting of —OH, —OP, wherein P is a protecting group, halo, alkyl, alkenyl, and alkynyl;

in the presence of an activator, a base, and optionally an additive.

According to some preferred embodiments of the present invention, Ar is phenyl or naphthyl. According to some preferred embodiments of the present invention, R⁴ and R⁵ are independently H or C₁ to C₆ alkyl

According to some embodiments of the present invention, R⁸ is alkyl or halo.

According to one embodiment of the present invention, the activator is selected from AOMP, AOP, BDDC, BDMP, BDP, BEC, BEMT, BEP, BEPH, BMP-Cl, BOMP, BOP, BOP-Cl, BroP, Bsmoc, Bspoc, Bts-Fmoc, BTFFH, BPMP, BTC, BTCFH (PyClU) Bts-Cl, CDMT, DCMT, DECP, DEPAT, DKP, DMCH, DPPAT, DOEPBI, DOPPBI, DPPBI, CC, CDPOP, CDPP, CF, CF₃-BOP CF₃-HBTU CF₃—NO₂-PyBOP, 6-Cl-HOBI, CF₃-PyBOP, 6-Cl-HOBt, CIC, CIP, CloP, CMBI, CMPI, COMU, Cpt-Cl, CPC, CPP, DCC, DEBP, DEPB, DEPBO, DEPBT, DEPC, DFIH, DIC, DEFFH, DMC, DMCH, DMCT, DMFFH, DMFH, DMTMM, DNAs, DNBs, DOMP, DOPBO, DOPBT, DPP-Cl, DPPA, EDC, FDMP, FDPP, FEP, FEPH, FIP, FOMP, HAE2PipU, HAE2PyU, HAM2PipU, HAM2PyU, HAMTU, HAMDU, HAPipU, HAPyU, HAPyTU, HAPTU, HATTU, HATU, HATeU, HBE2PipU, HBE2PyU, HBM2PipU, HBM2PyU, HBMTU, HBPTU, HBTeU, BMDU, HBPipU, HBPyU, HBTU, HDATU, HDAPyU, HDTU, HDATU, HDMA, 4-HDMA, HDMB, HDMC, 6-DMFB, HDMODC, HDMODeC, HDMOPC, HDMP, HDMPfp, HDmPyODC, HDPyU, HDTMA, HDTMB, HDmPyODeC, HDmPyOC, HMPyODC, HMPyOC, HOAt, 4-HOAt, 5-HOAt, 6-HOAt, HOBI, HOBt, HOCt, HODhbt, HODhad, HODhat, HODT, HOSu, HOI, 6-NO₂-HOBt, HONP, HOPy, 6-CF₃-HOBt, PS—SO₂-HOBt, PS-HOSu, PS-DCT, HONB, HOTT, HOTT, HOTU, HPyOPfp, HPFTU, HPTU, HPyONP, HPyOTCp, HPySPfp, HSTU, HTODC, HTODeC, HTOPC, NAs, 2-NAs, 4-NAs, NBs, 2-NBs, 4-NBs, NDPP, N-HATU, N—CF₃-HBTU, N—CF₃-TBTU, N-HAPyU, N-HATTU, N-HBPyU, N-HBTU, N-TATU, N-TBTU, MPTA, MPTO, Mspoc, Mukaiyama's reagent, NDPP, NMM, NO₂-PyBOP, MSNT, Oxyma, PIC, PS-DCC, PS-EDC, PEC, PS-TBTU, PTF, PyAOP, PyBOP, PyBroP, PyCloP, PyDOP, PyCloK, PyPOP, PyDAOP, PyFOP, PyFNBOP, PyNOP, PyOxm, PyTOP, SOMP, TATU, TAs, TBs, TBCR1, TBCR2, TBCR3, TBTU, TDBTU, TCFH, TCP, TDATU, TDTU, TEFFH, TFMS-DEP, TFFH, TNTU, TODT, TOTT, TOTU, TPTU, TSTU, TOPPipU, T3P, TPFTU, TPhTU, and TPP.

In a preferred embodiment of the present invention, the activator is a uronium or phosphonium activator, preferably selected from AOP, BMP-Cl, BOMP, BOP, BOP-Cl, BroP, CF₃-BOP CF₃-HBTU CF₃—NO₂-PyBOP, ClOP, COMU, HAE2PipU, HAE2PyU, HAM2PipU, HAM2PyU, HAMTU, HAMDU, HAPipU, HAPyU, HAPyTU, HAPTU, HATTU, HATU, HATeU, HBE2PipU, HBE2PyU, HBM2PipU, HBM2PyU, HBMTU, HBPTU, HBTeU, BMDU, HBPipU, HBPyU, HBTU, HDATU, HDAPyU, HDTU, HDATU, HDPyU, HOTU, HPyOPfp, HPFTU, HPTU, HPyONP, HPyOTCp, HPySPfp, HSTU, HTODC, HTODeC, N-HATU, N—CF₃-HBTU, N—CF₃-TBTU, N-HAPyU, N-HATTU, N-HBPyU, N-HBTU, N-TATU, N-TBTU, NO₂-PyBOP, PS-TBTU, PTF, PyAOP, PyBOP, PyBroP, PyCloP, PyDOP, PyCloK, PyPOP, PyDAOP, PyFOP, PyFNBOP, PyNOP, PyOxm, PyTOP, TATU, TBTU, TDBTU, TDATU, TDTU, TNTU, TODT, TOTT, TOTU, TPTU, TSTU, TOPPipU, TPFTU, and TPhTU, as defined herein.

In some embodiments of the present invention, the base is selected from NR₃ wherein R can be H, alkyl, aryl, heteroaryl, alkenyl, alkynyl, benzyl or allyl; disilazanes; heterocyclic bases including DABCO, 1,5 diazobicyclo[4.3.0]non-5-ene, 1,8-diazabicyclo[5.4.0]undec-7-ene, DMAP, 2,6 lutidine, piperidine, pyrrole, 3-pyrroline, 2H-pyroole 2-pyrroline, pyrrolidine, carbazole, azaindole, isoindole, indole, 3-H indole, indolizine, indoline, pyridine, piperidine, quinuclidine 4-H quinolizine, isoquinoline, quinoline, 1,8 naphthyridine, tetrahydroquinoline, acridine, oxazole, isoxazole, benoxazole, benzothiazole, isothiazole, thiazole, benzimidazole, imidazole 2, imidazole, imidazolidine, tetrazole, 1,3,4-thiadiazole, 1,2,3-tetrazole, 1,2,4-triazole, benzotriazole, imidazolepyridines, indazole, oxadiazole, phenodiazene, thiomorpholine, dithiane, phenoxazine, morpholine, pyrazole, 2-pyrazoline, pyrazolidine, quinazoline, cinnoline, pyrimidine, pteridine, phthalazine, 1,2,4-triazline, 1,3,5-triazine, piperazine, quinoxaline, phenazine, 1H-indazole, pyridazine, hydantoins, cinnolines, cyclazines, triazolepyridines, 2,2,6,6-tetramethylpiperidine, 2,8,9-triisobutyl-2,5,8,9-tetraaza-1-phosphabicyclo[3.3.3]undecane, 2,8,9-triisopropyl-2,5,8,9-tetraaza-1-phosphabicyclo[3,3,3]undecane, 2,8,9-trimethyl-2,5,8,9-tetraaza-1-phosphabicyclo[3.3.3]undecane and substituted derivatives thereof; phosphazines where phosphazene base P₂—R, where R is R=alkyl, aryl, alkenyl, alkynyl, heteroaryl, benzyl, allyl, carbocyclic such as, 2-tert-butylamino-1-methyl-2-[tris(dimethylamino)phosphoranylidenamino]-perhydro-1,3,2-diazaphosphorinium iodide, 2-tert-butylimino-2-diethylamino-1,3-dimethylperhydro-1,3,2-diazaphosphorine, 1,1,1,3,3,3-hexakis(dimethylamino)diphosphazenium tetrafluoroborate, imino-tris(dimethylamino)phosphorane, 1,1,3,3,3-pentakis(dimethylamino)-1λ5,3λ5-diphosphazene 1-oxide phosphazene base P1-t-Bu, phosphazene base P4-t-Bu, phosphazene base P1-t-Bu-tris(tetramethylene), phosphazene base P2-Et, phosphazene base P1-t-Oct; metal alkoxide or hydroxide bases: M-OR where M=Be, Li, Na, Mg, K, Ca, Cs, Sc, Ti, Mg, Cu, Al and R═H, alkyl, aryl, alkenyl, alkynyl, heteroaryl, benzyl, allyl, carbocyclic; solid supported bases including 1,4-diazabicyclo[2.2.2]octane hydrochloride, polymer-bound, 1,8-diazabicyclo[5.4.0]undec-7-ene, polymer-bound, 2,6-di-tert-butylpyridine, polymer-bound, 4-(dimethylamino)pyridine, polymer-bound morpholine, polymer-bound, piperidine, polymer-bound, NR3 polymer-bound, where R═H, alkyl, aryl, heteroaryl, benzyl, allyl, alkenyl, or alkynyl, heteroaryl, carbocyclic, 2-tert-butylimino-2-diethylamino-1,3-dimethylperhydro-1,3,2-diazaphosphorine, polymer-bound, phosphazene base P2-t-Bu on polystyrene, 1,5,7-triazabicyclo[4.4.0]dec-5-ene bound to polystyrene; chiral bases including tetramisole, quinine, quinine acetate, quinidine gluconate, dihydroquinine, 9-epi-quinine, 3-hydroxy quinine, quinine N-oxide, hydroquinine 4-chlorobenzoate, hydroquinine-9-phenanthryl ether, quinidine, quinidine N-oxide, hydroquinidine, hydroquinidine 9-phenanthryl ether hydroquinidine 4-methyl-2-quinolyl ether, hydroquinine 4-methyl-2-quinolyl ether, O-desmethyl quinidine, hydroquinidine 4-chlorobenzoate, L-(−)-α-amino-ε-caprolactam hydrochloride, D-(+)-α-amino-ε-caprolactam hydrochloride, (R)-(−)-1-amino-2-propanol, (S)-(+)-1-amino-2-propanol, chiral amino acids, brucine, cinchonine, cinchonidine, dihydro-cinchonidine, dihydrocinchonine, O-methylcinchonidine, cinchonan-6′,9-diol, cinchonan-9-ol, (9S)-(±)-10,11-dihydro-6′-methoxy-cinchonan-9-ol, 7′-(trifluoromethyl)-10,11-dihydrocinchonan-9-ol, cupreine, β-isocupreidine, euprocin, ethylhydrocupreine, (+)-dehydroabietylamine, (+)-dehydroabietylamine, (S)-(−)-N,α-dimethylbenzylamine, ephedrine, pseudoephedrine, (S)-α-methyl-2-pyridinemethanol (R)-α-methyl-2-pyridinemethanol, strychnine, 2R,4S,5R)-2-hydroxymethyl-5-ethylquinuclidine, (2S,4S,5R)-2-aminomethyl-5-ethylquinuclidine, (2R,5R)-(+)-5-vinyl-2-quinuclidinemethanol, N-[3,5-bis(trifluoromethyl)phenyl]-N′-[(8a,9S)-10,11-dihydro-6′-methoxy-9-cinchonanyl]thiourea, N-[3,5-bis(trifluoromethyl)phenyl]-N′-[(9R)-6′-methoxy-9-cinchonanyl]thiourea, N-[3,5-bis(trifluoromethyl)phenyl]-N′-[(8a,9S)-6′-methoxy-9-cinchonanyl]thiourea, quinine ethyl carbonate, 9-acetoxyrubanone, (DHQD)2PHAL, (DHQ)2PHAL, (DHQD)2Pyr, (DHQ)2Pyr, (DHQD)2AQN and modifications thereof. Preferred bases include triethylamine, Hunig's base, DMAP, DBU or 1,8-diazabicyclo[5.4.0]undec-7-ene.

According to some embodiments of the present invention, the coupling of the phosphoramidate to the nucleoside is done in the presence of an activator, a base, and optionally an additive. Additives may be the same or different than the base. A preferred additive is a quinine or quinine derivative. However, any of the following are contemplated: NR₃ where R is H, alkyl, aryl, heteroaryl, alkenyl, alkynyl, benzyl or allyl; disilazanes; TMEDA, TMP; heterocyclic bases including DABCO, 1,5 diazobicyclo[4.3.0]non-5-ene, 1,8-diazabicyclo[5.4.0]undec-7-ene, DMAP, 2,6 lutidine, piperidine, pyrrole, 3-pyrroline, 2H-pyroole 2-pyrroline, pyrrolidine, carbazole, azaindole, isoindole, indole, 3-H indole, indolizine, indoline, pyridine, piperidine, quinuclidine 4-H quinolizine, isoquinoline, quinoline, 1,8 naphthyridine, tetrahydroquinoline, acridine, oxazole, isoxazole, benoxazole, benzothiazole, isothiazole, thiazole, benzimidazole, imidazole 2, imidazole, imidazolidine, tetrazole, 1,3,4-thiadiazole, 1,2,3-tetrazole, 1,2,4-triazole, benzotriazole, imidazolepyridines, indazole, oxadiazole, phenodiazene, thiomorpholine, dithiane, phenoxazine, morpholine, pyrazole, 2-pyrazoline, pyrazolidine, quinazoline, cinnoline, pyrimidine, pteridine, phthalazine, 1,2,4-triazline, 1,3,5-triazine, piperazine, quinoxaline, phenazine, 1H-indazole, pyridazine, hydantoins, cinnolines, cyclazines, triazolepyridines, 2,2,6,6-tetramethylpiperidine, 2,8,9-triisobutyl-2,5,8,9-tetraaza-1-phosphabicyclo[3.3.3]undecane, 2,8,9-triisopropyl-2,5,8,9-tetraaza-1-phosphabicyclo[3,3,3]undecane, 2,8,9-trimethyl-2,5,8,9-tetraaza-1-phosphabicyclo[3.3.3]undecane and substituted derivatives thereof; phosphazines where phosphazene base P₂—R, where R is R=alkyl, aryl, alkenyl, alkynyl, heteroaryl, benzyl, allyl, carbocyclic. This may include 2-tert-butylamino-1-methyl-2-[tris(dimethylamino)phosphoranylidenamino]-perhydro-1,3,2-diazaphosphorinium iodide, 2-tert-butylimino-2-diethylamino-1,3-dimethylperhydro-1,3,2-diazaphosphorine, 1,1,1,3,3,3-hexakis(dimethylamino)diphosphazenium tetrafluoroborate, imino-tris(dimethylamino)phosphorane, 1,1,3,3,3-pentakis(dimethylamino)-1λ5,3λ5-diphosphazene 1-oxide phosphazene base P1-t-Bu, phosphazene base P4-t-Bu, phosphazene base P1-t-Bu-tris(tetramethylene), phosphazene base P2-Et, phosphazene base P1-t-Oct; metal alkoxide or hydroxide bases: M-OR where M=Be, Li, Na, Mg, K, Ca, Cs, Sc, Ti, Mg, Cu, Al and R═H, alkyl, aryl, alkenyl, alkynyl, heteroaryl, benzyl, allyl, carbocyclic; solid supported bases including 1,4-diazabicyclo[2.2.2]octane hydrochloride, polymer-bound, 1,8-diazabicyclo[5.4.0]undec-7-ene, polymer-bound, 2,6-di-tert-butylpyridine, polymer-bound, 4-(dimethylamino)pyridine, polymer-bound morpholine, polymer-bound, piperidine, polymer-bound, NR3 polymer-bound, where R═H, alkyl, aryl, heteroaryl, benzyl, allyl, alkenyl, or alkynyl, heteroaryl, carbocyclic, 2-tert-butylimino-2-diethylamino-1,3-dimethylperhydro-1,3,2-diazaphosphorine, polymer-bound, phosphazene base P2-t-Bu on polystyrene, 1,5,7-triazabicyclo[4.4.0]dec-5-ene bound to polystyrene; chiral bases including tetramisole, quinine and quinine derivatives such as quinine acetate, quinidine gluconate, dihydroquinine, 9-epi-quinine, 3-hydroxy quinine, quinine N-oxide, hydroquinine 4-chlorobenzoate, hydroquinine-9-phenanthryl ether, quinidine, quinidine N-oxide, hydroquinidine, hydroquinidine 9-phenanthryl ether hydroquinidine 4-methyl-2-quinolyl ether, hydroquinine 4-methyl-2-quinolyl ether, 0-desmethyl quinidine, hydroquinidine 4-chlorobenzoate, L-(−)-α-amino-ε-caprolactam hydrochloride, D-(+)-α-amino-ε-caprolactam hydrochloride, (R)-(−)-1-amino-2-propanol, (S)-(+)-1-amino-2-propanol, chiral amino acids, brucine, cinchonine, cinchonidine, dihydro-cinchonidine, dihydrocinchonine, O-methylcinchonidine, cinchonan-6′,9-diol, cinchonan-9-ol, (9S)-(±)-10,11-dihydro-6′-methoxy-cinchonan-9-ol, 7′-(trifluoromethyl)-10,11-dihydrocinchonan-9-ol, cupreine, β-isocupreidine, euprocin, ethylhydrocupreine, (+)-dehydroabietylamine, (+)-dehydroabietylamine, (S)-(−)-N,α-dimethylbenzylamine, ephedrine, pseudoephedrine, (S)-α-methyl-2-pyridinemethanol (R)-α-methyl-2-pyridinemethanol, strychnine, 2R,4S,5R)-2-hydroxymethyl-5-ethylquinuclidine, (2S,4S,5R)-2-aminomethyl-5-ethylquinuclidine, (2R,5R)-(+)-5-vinyl-2-quinuclidinemethanol, N-[3,5-bis(trifluoromethyl)phenyl]-N′-[(8a,9S)-10,11-dihydro-6′-methoxy-9-cinchonanyl]thiourea, N-[3,5-bis(trifluoromethyl)phenyl]-N′-[(9R)-6′-methoxy-9-cinchonanyl]thiourea, N-[3,5-bis(trifluoromethyl)phenyl]-N′-[(8a,9S)-6′-methoxy-9-cinchonanyl]thiourea, quinine ethyl carbonate, 9-acetoxyrubanone, (DHQD)2PHAL, (DHQ)2PHAL, (DHQD)2Pyr, (DHQ)2Pyr, (DHQD)2AQN and modifications thereof.

According to one preferred embodiment of the present invention, a process for making a compound having the following Formula IV is provided:

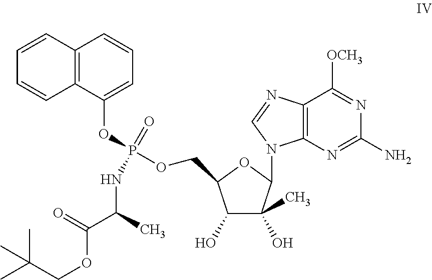 [SMILES: COc1nc(N)nc2c1ncn2C1O[C@H](CO[P@@](=O)(N[C@@H](C)C(=O)OCC(C)(C)C)Oc2cccc3ccccc23)[C@@H](O)[C@@]1(C)O]

comprising contacting a compound having Formula V:

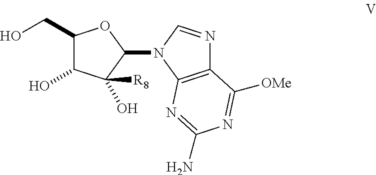

with a compound having Formula VI, or a salt thereof:

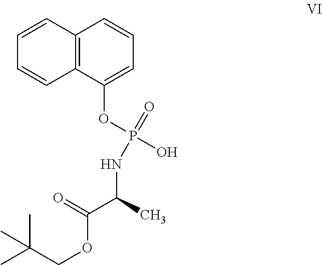 [SMILES: C[C@H](NP(=O)(O)Oc1cccc2ccccc12)C(=O)OCC(C)(C)C]

in the presence of an activator, such as a phosphonium or uronium activator, a base, such as Hunig's base and optionally an additive, such as quinine or a quinine derivative.

According to one embodiment of the present invention, a process for making a compound having the following Formula X is presented:

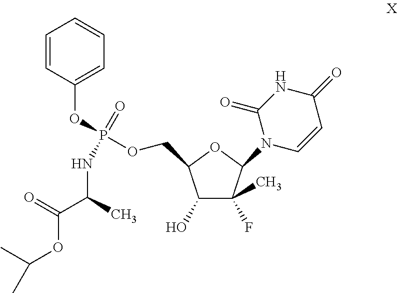 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

comprising contacting a compound having the Formula XI:

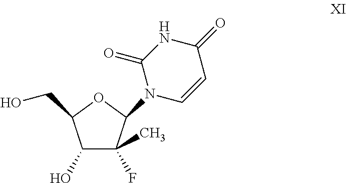 [SMILES: C[C@@]1(F)[C@H](O)[C@@H](CO)O[C@H]1n1ccc(=O)[nH]c1=O]

with a compound having the Formula XII

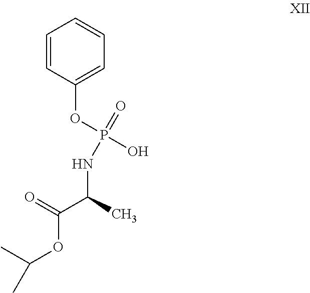 [SMILES: CC(C)OC(=O)[C@H](C)NP(=O)(O)Oc1ccccc1]

or a salt thereof in the presence of an activator, a base, and an optional additive, such as those described herein.

According to one embodiment of the present invention, a compound having the following Formula II is provided:

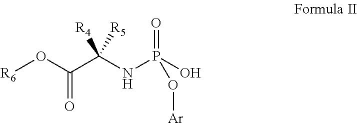

wherein

R⁴ and R⁵ are independently selected from hydrogen, C₁-C₆alkyl optionally substituted with alkylthio, benzyl optionally substituted with one or more halo, C₁-C₆alkyl, or C₁-C₆alkoxy, phenyl optionally substituted with one or more halo, C₁-C₆alkyl, or C₁-C₆alkoxy;

R⁶ is selected from C₁-C₁₀alkyl, C₃-C₈cycloalkyl, C₃-C₈cycloalkyl-alkyl-, phenyl(C₁-C₆)alkyl- optionally substituted with C₁-C₆alkyl, C₁-C₆alkoxy, and halo, indanyl and heterocycloalkyl; and

Ar is selected from phenyl, naphthyl,

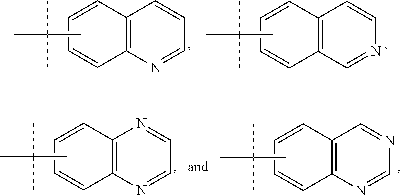

any of which are optionally substituted with C₁-C₆alkyl, C₁-C₆alkoxy, di(C₁-C₆)alkylamino or C₁-C₆alkylcarboxy(C₁-C₆)alkyl-.

Compounds of Formula II may form salts, hydrates or solvates by way of example only, calcium, zinc, DBU or Hunig's base salts, as neat forms, hydrates or solvates.

According to one embodiment of the present invention, a compound having the following Formula XII is provided:

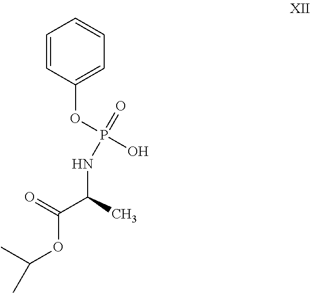 [SMILES: CC(C)OC(=O)[C@H](C)NP(=O)(O)Oc1ccccc1]

and may exist as a salt, hydrate or solvate, such as by way of example only, calcium, zinc, DBU or Hunig's base salts, as neat forms, hydrates or solvates.

While the invention has been described with reference to particularly preferred embodiments and examples, those skilled in the art recognize that various modifications may be made to the invention without departing from the spirit and scope thereof

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Greene, T. W. et al., Protecting Groups in Organic Synthesis, Third Edition, Wiley, New York (1999), and references cited therein.

The following acronyms as used herein are defined as follows:

| | |
|---|---|
| AOMP | 5-(7-azabenzotriazol-1-yloxy)-3,4-dihydro-1-methyl 2H-pyrrolium |
| | hexachloroantimonate |
| AOP | (7-azabenzotriazol-1-yl)oxytris(dimethylamino)phosphonium |
| | hexafluorophosphate |
| BDDC | bis[[4-(2,2-dimethyl-1,3-dioxolyl)]-methyl]-carbodiimide |
| BDMP | 5-(1H-benzotriazol-1-yloxy)-3,4-dihydro-1-methyl 2H-pyrrolium |
| | hexachloroantimonate |
| BDP | benzotriazol-1-yl diethylphosphate |
| BEC | N-tert-butyl-N′-ethylcarbodiimide |
| BEMT | 2-bromo-3-ethyl-4-methyl thiazolium tetrafluoroborate |
| BEP | 2-bromo-1-ethyl pyridinium tetrafluoroborate |
| BEPH | 2-bromo-1-ethyl pyridinium hexachloroantimonate |
| BMP-Cl | N,N′-bismorpholinophosphinic chloride |
| Boc | t-butyloxycarbonyl |
| BOMP | 2-(benzotriazol-1-yloxy)-1,3-dimethyl-2-pyrrolidin-1-yl-1,3,2- |
| | diazaphospholidinium hexafluorophosphate |
| BOP | benzotriazol-1-yloxytris(dimethylamino)phosphonium |
| | hexafluorophosphate |
| BOP-Cl | N,N′-bis(2-oxo-3-oxazolidinyl)phosphinic chloride |
| BroP | bromotris(dimethylamino)phosphoniumhexafluorophosphate |
| Bsmoc | 1,1-dioxobenzo[b]thiophene-2-ylmethyloxycarbonyl |
| Bspoc | 2-(tert-butylsulfonyl)-2-propyloxycarbonyl |
| Bts-Fmoc | 2,7-bis(trimethylsilyl)-9-fluorenylmethyloxycarbonyl |
| BTFFH | bis(tetramethylene)fluoroformamidiniumhexafluorophosphate |
| BPMP | 1-(1H-benzotriazol-1-yloxy)phenylmethylene pyrrolidinium |
| | hexachloroantimonate |
| BTC | triphosgene |
| BTCFH | bis(tetramethylene)chlororformamidinium hexafluorophosphate |
| (PyClU) | |
| Bts-Cl | benzothiazol-2-sulfonyl chloride |
| Cbz, Z | benzyloxycarbonyl |
| CDMT | 2-chloro-4,6-dimethoxy-1,3,5-triazine |
| DCMT | 2,4-dichloro-6-methoxy-1,3,5-triazine |
| DECP | diethylcyanophosphonate |
| DEPAT | 3-(diethoxyphosphoryloxy)-1,2,3-pyridino-[b]triazin-4-(3H)-one |
| DMCH | N-(chloro(morpholino)methylene)-N-methylmethanaminium |
| | hexafluorophosphate |
| DPPAT | 3-(diphenoxyphosphoryloxy)-1,2,3-pyridino-[b]triazin-4-(3H)-one |
| DOEPBI | phosphoric acid diethyl ester 2-phenylbenzimidazol-1-yl ester |
| DOPPBI | phosphoric acid diphenyl-2-phenylbenzimidazol-1-yl ester |
| DPPBI | diphenylphosphinic acid 2-phenylbenzimidazol-1-yl ester |
| CC | cyanuric chloride |
| CDPOP | pentachlorophenyl diphenyl phosphate |
| CDPP | pentachlorophenyl diphenyl phosphinate |
| CF | cyanuric fluoride |
| CF₃-BOP | [6-(trifluoromethyl)benzotriazol-1-yl]-Noxy- |
| | tris(dimethylamino)phosphonium hexafluorophosphate |
| CF₃-HBTU | 2-[6-(trifluoromethyl)-benzotriazol-1-yl]-1,1,3,3-tetramethyluronium |
| | hexafluorophosphate |
| CF₃—NO₂- | [4-nitro-6-(trifluoromethyl)benzotriazol-1-yl)-oxy]tris(pyrrolidino) |
| PyBOP | |
| 6-Cl-HOBI | 6-chloro-N-hydroxy-2-phenylbenzimidazole phosphonium |
| | hexafluorophosphate |
| CF₃-PyBOP | [6-(trifluoromethyl)-benzotriazol-1-yl]-N-oxytris- |
| | (pyrrolidino)phosphonium hexafluorophosphate |
| 6-Cl-HOBt | 6-chloro-1-hydroxybenzotriazole |
| CIC | N-cyclohexyl,N′-isopropyl carbodiimide |
| CIP | 2-chloro-1,3-dimethylimidazolidinium hexafluorophosphate |
| CloP | chloro-tris(dimethylamino)phosphoniumhexafluorophosphate |
| CMBI | 2-chloro-1,3-dimethyl 1H-benzimidazolium hexafluorophosphate |
| CMPI | 2-chloro-1-methylpyridinium iodide |
| COMU | 1-[(1-(cyano-2-ethoxy-2-oxoethylideneaminooxy)-dimethylamino- |
| | morpholinomethylene)]methanaminium hexafluorophosphate |
| Cpt-Cl | 1-oxo-chlorophospholane |
| CPC | N,N′-dicyclopentylcarbodiimide |
| CPP | 2-chloro-1,3-dimethylpyrimidiniumhexafluorophosphate |
| DCC | N,N′-dicyclohexylcarbodiimide |
| DEBP | diethyl 2-(3-oxo-2,3-dihydro-1,2-benzisosulfonazolyl)phosphonate |
| DEPB | diethyl phosphorobromidate |
| DEPBO | N-diethoxyphosphoryl benzoxazolone |
| DEPBT | 3-(diethoxyphosphoryloxy)-1,2,3-benzotriazin-4(3H)-one |
| DEPC | diphenyl phosphorochloridate |
| DFIH | 1,3-dimethyl-2-fluoro-4,5-dihydro-1H-imidazolium |
| | hexafluorophosphate |
| DIC | N,N′-diisopropylcarbodiimide |
| DEFFH | 1,2-diethyl-3,3-tetramethylne fluoroformamidinium |
| | hexafluorophosphate |
| DMCH | N-(chloro(morpholino)methylene)-N-methylmethanaminium |
| | hexafluorophosphate |
| DMCT | 2-chloro-4,6-dimethyl-1,3,5-triazine |
| DMFFH | 1,2-dimethyl-3,3-tetramethylene fluoroformamidinium |
| | hexafluorophosphate |
| DMFH | N-(fluoro(morpholino)methylene)-N-methylmethanaminium |
| | hexafluorophosphate |
| DMTMM | 4-(4,6-dimethoxy[1,3,5]triazin-2-yl)-4-methylmorpholinium chloride |
| DNAs | 3H-[1,2,3]triazolo[4,5-b]pyridin-3-yl 2,4-dinitrobenzenesulfonate |
| DNBs | 1H-benzo[d][1,2,3]triazol-1-yl 2,4-dinitrobenzenesulfonate |
| DOMP | 5-(30,40-dihydro-40-oxo-10,20,30-benzotriazin-30-yloxy)-3,4-dihydro- |
| | 1-methyl-2H-pyrrolium hexachloroantimonate |
| DOPBO | N-(2-oxo-1,3,2-dioxaphosphorinanyl)-benzoxazolone |
| DOPBT | 3-[O-(2-oxo-1,3,2-dioxaphosphorinanyl)-oxy]-1,2,3-benzotriazin- |
| | 4(3H)-one |
| DPP-Cl | diphenylphosphinic chloride |
| DPPA | diphenylphosphoryl azide |
| Dtb-Fmoc | 2,7-di-tert-butyl-9-fluorenylmethyloxycarbonyl |
| EDC | 1-[3-(dimethylamino)propyl]-3-ethylcarbodiimide hydrochloride |
| FDMP | 3,5-bis(trifluoromethylphenyl)phenyl diphenylphosphinate |
| FDPP | pentafluorophenyl diphenyl phosphinate |
| FEP | 2-fluoro-1-ethyl pyridinium tetrafluoroborate |
| FEPH | 2-fluoro-1-ethyl pyridinium hexachloroantimonate |
| FIP | 2-fluoro-1,3-dimethylimidazolidiniumhexafluorophosphate |
| Fmoc | 9-fluorenylmethyloxcarbonyl |
| FOMP | 5-(pentafluorophenyloxy)-3,4-dihydro-1-methyl- |
| 2H-pyrrolium | hexachloroantimonate |
| HAE2PipU | O-(1H-1,2,3-triazolo[4,5-b]pyridin-1-yl)-1,1-diethyl-3,3- |
| | pentamethyleneuronium |
| HAE2PyU | O-(1H-1,2,3-triazolo[4,5-b]pyridin-1-yl)-1,1-diethyl-3,3- |
| | tetramethyleneuronium hexafluorophosphate |
| HAM2PipU | O-(1H-1,2,3-triazolo[4,5-b]pyridin-1-yl)-1,1-dimethyl-3,3- |
| | pentamethyleneuronium hexafluorophosphate |
| HAM2PyU | O-(1H-1,2,3-triazolo[4,5-b]pyridin-1-yl)-1,1-dimethyl-3,3- |
| | tetramethyleneuronium hexafluorophosphate |
| HAMTU | O-(7-azabenzotriazol-1-yl)-1,3-dimethyl-1,3-trimethyleneuronium |
| | hexafluorophosphate hexafluorophosphate |
| HAMDU | O-(7-azabenzotriazol-1-yl)-1,3-dimethyl-1,3-dimethyleneuronium |
| | hexafluorophosphate |
| HAPipU | O-(7-azabenzotriazol-1-yl)-1,1,3,3-bis(pentamethylene)uronium |
| | hexafluorophosphate |
| HAPyU | 1-(1-pyrrolidinyl-1H-1,2,3-triazolo[4,5-b]-pyridin-1- |
| | ylmethylene)pyrrolidinmium hexafluorophosphate N-oxide |
| HAPyTU | 1-(1-pyrrolidinyl-1H-1,2,3-triazolo[4,5-b]-pyridin-1- |
| | ylmethylene)pyrrolidinmium hexafluorophosphate N-sulfide |
| HAPTU | (7-azabenzotriazol-yl)-1,1,3-trimethyl-1-phenyluronium |
| | hexafluorophosphate |
| HATTU | S-(7-azabenzotriazol-1-yl)-1,1,3,3-tetramethyluronium |
| | hexafluorophosphate |
| HATU | O-(7-azabenzotriazol-1-yl)-1,1,3,3-tetramethyluronium |
| | hexafluorophosphate |
| HATeU | O-(1H-1,2,3-triazolo[4,5-b]pyridin-1-yl)-1,1,3,3-tetraethyluronium |
| | hexafluorophosphate |
| HBE2PipU | O-(1H-benzotriazol-1-yl)-1,1-diethyl-3,3-pentamethyleneuronium |
| | hexafluorophosphate |
| HBE2PyU | O-(1H-benzotriazol-1-yl)-1,1-diethyl-3,3-tetramethyleneuronium |
| | hexafluorophosphate |
| HBM2PipU | O-(1H-benzotriazol-1-yl)-1,1-dimethyl-3,3-pentamethyleneuronium |
| | hexafluorophosphate |
| HBM2PyU | O-(1H-benzotriazol-1-yl)-1,1-dimethyl-3,3-tetramethyleneuronium |
| | hexafluorophosphate |
| HBMTU | O-(benzotriazol-1-yl)-1,3-dimethyl-1,3-trimethyleneuronium |
| | hexafluorophosphate |
| HBPTU | (7-benzotriazol-yl)-1,1,3-trimethyl-1-phenyluronium |
| | hexafluorophosphate |
| HBTeU | O-(1H-benzotriazol-1-yl)-1,1,3,3-tetraethyluronium |
| | hexafluorophosphate |
| HBMDU | O-(benzotriazol-1-yl)-1,3-dimethyl-1,3-dimethyleneuronium |
| | hexafluorophosphate |
| HBPipU | O-(benzotriazol-1-yl)-1,1,3,3-bis(pentamethylene)uronium |
| | hexafluorophosphate |
| HBPyU | O-(benzotriazol-1-yl)oxybis(pyrrolidino)-uronium hexafluorophosphate |
| HBTU | O-(benzotriazol-1-yl)-1,1,3,3-tetramethyluronium hexafluorophosphate |
| HDATU | O-(3,4-dihydro-4-oxo-5-azabenzo-1,2,3-triazin-3-yl)-1,1,3,3- |
| | tetramethyluronium hexafluorophosphate |
| HDAPyU | O-(3,4-dihydro-4-oxo-5-azabenzo-1,2,3-triazin-3-yl)-1,1,3,3- |
| | bis(tetramethylene)uronium hexafluorophosphate |
| HDTU | O-(3,4-dihydro-4-oxo-1,2,3-benzotriazin-3-yl)-1,1,3,3- |
| | tetramethyluronium hexafluorophosphate |
| HDATU | O-(3,4-dihydro-4-oxo-5-azabenzo-1,2,3-triazin-3-yl)-1,1,3,3- |
| | tetramethyluronium hexafluorophosphate |
| HDMA | 1-((dimethylamino)-(morpholino)methylene)-1H-[1,2,3]triazolo[4,5- |
| | b]pyridinium hexafluorophosphate-3-oxide |
| 4-HDMA | 3-((dimethylamino)-(morpholino)methylene)-1H-[1,2,3]triazolo[4,5- |
| | b]pyridinium hexafluorophosphate-1-oxide |
| HDMB | 1-((dimethylamino)(morpholino)methylene)-1H-benzotriazolium |
| | hexafluorophosphate-3-oxide |
| HDMC | 6-chloro-1-((dimethylamino)(morpholino)-methylene)-1H- |
| | benzotriazolium hexafluorophosphate-3-oxide |
| 6-HDMFB | 6-trifluoromethyl-1-((dimethylamino)(morpholino)methylene)-1H- |
| | benzotriazolium hexafluorophosphate-3-oxide |
| HDMODC | 1-[(1-(dicyanomethyleneaminooxy)- |
| | dimethylaminomorpholinomethylene)]methanaminium |
| | hexafluorophosphate |
| HDMODeC | 1-[(1,3-diethyoxy-1,3-dioxopropan-2-ylideneaminooxy)- |
| | dimethylamino-morpholinomethylene)]methanaminium |
| | hexafluorophosphate |
| HDMOPC | N-[(cyano(pyridine-2-yl)methyleneaminooxy)- |
| | (dimethylamino)methylene]-N-morpholinomethanaminium |
| | hexafluorophosphate |
| HDMP | 1-((dimethylamino)(morpholino))oxypyrrolidine-2,5-dione |
| | methanaminium hexafluorophosphate |
| HDMPfp | 1-((dimethylamino)- |
| | (morpholino))oxypentafluorophenylmetheniminium |
| | hexafluorophosphate |
| HDmPyODC | 1-[(1-(cyano-2-ethoxy-2-oxoethylideneaminooxy)- |
| | dimethylaminopyrrolodino methylene)]methanaminium |
| | hexafluorophosphate |
| HDPyU | O-(3,4-dihydro-4-oxo-1,2,3-benzotriazin-3-yl)-1,1,3,3- |
| | bis(tetramethylene)uronium hexafluorophosphate |
| HDTMA | 1-((dimethylamino)(thiomorpholino)methylene)-1H-[1,2,3]triazolo[4,5- |
| | b]pyridinium hexafluorophosphate-3-oxide |
| HDTMB | 1-((dimethylamino)(thiomorpholino)methylene)-1H-benzotriazolium |
| | hexafluorophosphate-3-oxide |
| HDmPyODeC | 1-[(1,3-diethyoxy-1,3-dioxopropan-2-ylideneaminooxy)-dimethylamino |
| | pyrrolodinomethylene)]methanaminium hexafluorophosphate |
| HDmPyOC | 1-[(1-(cyano-2-ethoxy-2-oxoethylideneaminooxy)-dimethylamino- |
| | pyrrolodinomethylene)]methanaminium hexafluorophosphate |
| HMPyODC | 1-((dicyanomethyleneaminooxy)morpholinomethylene)pyrrolidinium |
| | hexafluorophosphoate |
| HMPA | hexamethylphosphoramide |
| HMPyOC | 1-((1-cyano-2-ethoxy-2- |
| | oxoethylideneaminooxy)(morpholino)methylene)pyrrolidinium |
| | hexafluorophosphate |
| HOAt | 1-hydroxy-7-azabenzotriazole |
| 4-HOAt | 4-aza-1-hydroxybenzotriazole |
| 5-HOAt | 5-aza-1-hydroxybenzotriazole |
| 6-HOAt | 6-aza-1-hydroxybenzotriazole |
| HOBI | N-hydroxy-2-phenylbenzimidazole |
| HOBt | 1-hydroxybenzotriazole |
| HOCt | ethyl-1-hydroxy-1H-1,2,3-triazole-4-carboxylate |
| HODhbt | 3,4-dihydro-3-hydroxy-4-oxo-1,2,3-benzotriazine |
| HODhad | 3-hydroxy-4-oxo-3,4-dihydro-5-azabenzo-1,3-diazene |
| HODhat | 3-hydroxy-4-oxo-3,4-dihydro-5-azabenzo-1,2,3-triazene |
| HODT | S-(1-oxido-2-pyridinyl)-1,3-dimethyl-1,3-trimethylenethiouronium |
| | hexafluorophosphate |
| HOSu | N-hydroxysuccinimide |
| HOI | N-hydroxyindolin-2-one |
| 6-NO₂-HOBt | 1-hydroxy-6-nitrobenzotriazole |
| HONP | p-nitrophenyl active ester |
| HOPy | 1-hydroxy-2-pyridinone |
| 6-CF₃-HOBt | 6-trifluoromethyl-1-hydroxy benzotriazole |
| PS-SO₂-HOBt | polymer-supported 1-hydroxy-6-disulfoxide benzotriazole |
| PS-HOSu | polymer-supported N-hydroxysuccinimide |
| PS-DCT | polymer-supported 2,4-dichloro-1,3,5-triazine |
| HONB | N-hydroxy-5-norbornene-endo-2,3-dicarboxyimide |
| HOTT | S-(1-oxido-2-pyridinyl)-1,1,3,3-tetramethylthiouronium |
| | hexafluorophosphate |
| HOTT | S-(1-oxido-2-pyridinyl)-1,1,3,3-tetramethylthiouronium |
| | hexafluorophosphate |
| HOTU | O-[cyano(ethoxycarbonyl)methyleneamino]-N,N,N′,N′- |
| | tetramethyluronium hexafluorophosphate |
| HPyOPfp | N,N,N′,N′-bis(tetramethylene)-O-pentafluorophenyluronium |
| | hexafluorophosphate |
| HPFTU | N,N,N′,N′-bis(tetramethylene)-O-pentafluorophenyluronium |
| | hexafluorophosphate |
| HPTU | 2-(2-oxo-1(2H)-pyridyl-1,1,3,3-tetramethyluronium |
| | hexafluorophosphate |
| HPyONP | N,N,N′,N′-bis(tetramethylene)-O-2-nitrophenyluronium |
| | hexafluorophosphate |
| HPyOTCp | N,N,N′,N′-bis(tetramethylene)-O-pentafluorophenyluronium |
| | hexafluorophosphate |
| HPySPfp | N,N,N′,N′-bis(tetramethylene)-S-pentafluorothiophenyluronium |
| | hexafluorophosphate |
| HSTU | 2-succinimido-1,1,3,3-tetramethyluroniumhexafluorophosphate |
| HTODC | O-[(dicyanomethylidene)-amino]-1,1,3,3-tetramethyluronium |
| | hexafluorophosphate |
| HTODeC | O-[(diethoxycarbonylmethylidene)amino]-1,1,3,3-tetramethyluronium |
| | hexafluorophosphate |
| HTOPC | N-[(cyano(pyridine-2-yl)methyleneaminooxy)- |
| | (dimethylamino)methylene)-N-methyl methanaminium |
| | hexafluorophosphate |
| NAs | 3-((naphthalen-2-ylsulfonyl)methyl)-3H-[1,2,3]-triazolo[4,5-b]pyridine |
| 2-NAs | 3H-[1,2,3]triazolo[4,5-b]pyridin-3-yl 2-nitrobenzenesulfonate |
| 4-NAs | 3H-[1,2,3]triazolo[4,5-b]pyridin-3-yl 4-nitrobenzenesulfonate |
| NBs | 1-((naphthalen-2-ylsulfonyl)methyl)-1H-benzo-[d][1,2,3]triazole |
| 2-NBs | 1H-benzo[d][1,2,3]triazol-1-yl 2-nitrobenzenesulfonate |
| 4-NBs | 1H-benzo[d][1,2,3]triazol-1-yl 4-nitrobenzenesulfonate |
| NDPP | norborn-5-ene-2,3-dicarboximidodiphenylphosphate |
| N-HATU | N-[(dimethylamino)-1H-1,2,3-triazolo[4,5-b]-pyridin-1-ylmethylene]- |
| | N-methylmethanaminium hexafluorophosphate N-oxide |
| N-CF₃-HBTU | N-[6-trifluoromethyl(1H-benzotriazol-1-yl)- |
| | (dimethylamino)methylene]-N-methylmethanaminium |
| | hexafluorophosphate N-oxide |
| N-CF₃-TBTU | N-[6-trifluoromethyl(1H-benzotriazol-1-yl)- |
| | (dimethylamino)methylene]-N-methylmethanaminium tetrafluoroborate |
| | N-oxide |
| N-HAPyU | 1-(1-pyrrolidinyl-1H-1,2,3-triazolo[4,5-b]pyridin-1- |
| | ylmethylene)pyrrolidinium hexafluorophosphate N-oxide |
| N-HATTU | N-[(dimethylamino)-1H-1,2,3-triazolo[4,5-b]-pyridin-1-ylmethylene]- |
| | N-methylmethanaminium hexafluorophosphate N-sulfide |
| N-HBPyU | (1H-benzotriazol-1-yl)(1-pyrrolidinylmethylene)pyrrolidinium |
| | hexafluorophosphate N-oxide |
| N-HBTU | N-[(1H-benzotriazol-1-yl)(dimethylamino)-methylene]-N- |
| | methylmethanaminium hexafluorophosphate N-oxide |
| N-TATU | N-[(dimethylamino)-1H-1,2,3-triazolo[4,5-b]-pyridin-1-ylmethylene]- |
| | N-methylmethanaminium tetrafluoroborate N-oxide |
| N-TBTU | N-[(1H-benzotriazol-1-yl)(dimethylamino)-methylene]-N- |
| | methylmethanaminium tetrafluoroborate N-oxide |
| MPTA | dimethylphosphinothioyl azide |
| MPTO | 3-dimethylphosphinothioyl-2(3H)-oxazolone |
| Mspoc | 2-methylsulfonyl-3-phenyl-1-prop-2-enyloxycarbonyl |
| Mukaiyama's | 2-chloro-1-methylpyridinium iodide |
| reagent | |
| NDPP | norborn-5-ene-2,3-dicarboximidodiphenylphosphate |
| NMM | N-methylmorpholine |
| NO₂-PyBOP | (6-nitrobenzotriazol-1-yloxy)tris(pyrrolidino)phosphonium |
| | hexafluorophosphate |
| Oxyma | ethyl 2-cyano-2-(hydroxyimino)acetate |
| PIC | N-phenyl,N-isopropylcarbodiimide |
| PS | polymer supported |
| PS-DCC | polymer cyclohexylcarbodiimide |
| PS-EDC | polymer 1-ethyl-3-(30-dimethylaminopropyl)-carbodiimide |
| PEC | N-ethyl, N-phenylcarbodiimide |
| PS-TBTU | N-[(1H-benzotriazol-1-yl)(dimethylamino)-methylene]-N- |
| | methylmethanaminium tetrafluoroborate N-oxide |
| PTF | benzyltriphenylphosphonium dihydrogen trifluoride |
| PyAOP | [(7-azabenzotriazol-1-yl)oxy]tris(pyrrolidino)phosphonium |
| | hexafluorophosphate |
| PyBOP | benzotriazol-1-yloxytri(pyrrolidino)phosphonium hexafluorophosphate |
| PyBroP | bromotri(pyrrolidino)phosphonium hexafluorophosphate |
| PyCloP | chlorotri(pyrrolidino)phosphoniumhexafluorophosphate |
| PyDOP | [(3,4-dihydro-4-oxo-1,2,3-benzotriazin-3-yl)oxy]- |
| | tris(pyrrolidino)phosphonium hexafluorophosphate |
| PyCloK | (6-chloro-benzotriazol-1-yloxy)tris(pyrrolidino)phosphonium |
| | hexafluorophosphate |
| PyPOP | (pentafluorophenyloxy)tris(pyrrolidino)phosphonium |
| | hexafluorophosphate |
| PyDAOP | [(3,4-dihydro-4-oxo-5-azabenzo-1,2,3-triazin-3- |
| | yl]tris(pyrrolidino)phosphonium hexafluorophosphate |
| PyFOP | [[6-(trifluoromethyl)benzotriazol-1-yl]oxy]- |
| | tris(pyrrolidino)phosphonium hexafluorophosphate |
| PyFNBOP | [4-nitro-6-(trifluoromethyl)benzotriazol-1-yl)- |
| | oxy]tris(pyrrolidino)phosphonium hexafluorophosphate |
| PyNOP | [(6-nitrobenzotriazol-1-yl)oxy]tris(pyrrolidino)phosphonium |
| | hexafluorophosphate |
| PyOxm | O-[(cyano(ethoxycarbonyl)methyliden)-amino]- |
| | yloxytri(pyrrolidino)phosphonium hexafluorophosphate |
| PyTOP | (pyridyl-2-thio)tris(pyrrolidino)phosphonium hexafluorophosphate |
| SOMP | 5-(succinimidyloxy)-3,4-dihydro-1-methyl 2H-pyrrolium |
| | hexachloroantimonate |
| TATU | O-(7-azabenzotriazol-1-yl)-1,1,3,3-tetramethyluronium |
| | tetrafluoroborate |
| TAs | 3H-[1,2,3]triazolo[4,5-b]pyridin-3-yl 4-methylbenzenesulfonate |
| TBs | 1H-benzo[d][1,2,3]triazol-1-yl 4-methylbenzenesulfonate |
| TBCR1 | 4-(4,6-dimethoxy-1,3,5-triazin-2-yl)-4-methylmorpholinium |
| | tetrafluoroborate |
| TBCR2 | 1-(4,6-dimethoxy-1,3,5-triazin-2-yl)-1-methylpiperydinium |
| | tetrafluoroborate |
| TBCR3 | 1-(4,6-dimethoxy-1,3,5-triazin-2-yl)quinuclidinium tetrafluoroborate |
| TBTU | O-benzotriazol-1-yl-1,1,3,3-tetramethyluronium tetrafluoroborate |
| TDBTU | 2-(3,4-dihydro-4-oxo-1,2,3-benzotriazin-3-yl)-1,1,3,3- |
| | tetramethyluronium tetrafluoroborate |
| TCFH | tetramethylchloroformamidinium hexafluorophosphate |
| TCP | 2,4,5-trichlorophenyl active ester |
| TDATU | O-(3,4-dihydro-4-oxo-5-azabenzo-1,2,3-triazin-3-yl)-1,1,3,3- |
| | tetramethyluronium tetrafluoroborate |
| TDTU | 2-(3,4-dihydro-4-oxo-1,2,3-benzotriazin-3-yl)-1,1,3,3- |
| | tetramethyluronium tetrafluoroborate |
| TEFFH | tetraethylfluoroformamidinium hexafluorophosphate |
| TFMS-DEP | diphenyl(trifluoromethylsulfonyl)phosphoramidate |
| TFFH | tetramethylfluoroformamidiniumhexafluorophosphate |
| TNTU | 2-(5-norbornene-2,3-dicarboximido)-1,1,3,3-tetramethyluronium |
| | tetrafluoroborate |
| TODT | S-(1-oxido-2-pyridinyl)-1,3-dimethyl-1,3-trimethylenethiouronium |
| | tetrafluoroborate |
| TOTT | S-(1-oxido-2-pyridinyl)-1,1,3,3-tetramethylthiouronium |
| | tetrafluoroborate |
| TOTU | O-[cyano(ethoxycarbonyl)methyleneamino]-N,N,N′,N′- |
| | tetramethyluronium tetrafluoroborate |
| TPTU | 2-(2-oxo-1(2H)-pyridyl-1,1,3,3-tetramethyluronium tetrafluoroborate |
| TSTU | 2-succinimido-1,1,3,3-tetramethyluroniumtetrafluoroborate |
| TOPPipU | 2[2-Oxo-1(2H)-pyridyl]-1,1,3,3-bis(pentamethylene)uronium |
| | tetrafluoroborate |
| T3P; PPAA | 2-propanephosphonic acid anhydride |
| TPFTU | N,N,N′,N′-bis(tetramethylene)-O-pentafluorophenyluronium |
| | tetrafluoroborate |
| TPhTU | 2-phthalimido-1,1,3,3-tetramethyluronium tetrafluoroborate |
| TPP | triphenylphosphine carbon tetrachloride |

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15, John Wiley and Sons (1991), Rodd's Chemistry of Carbon Compounds, Volumes 1-5 and Supplementals, Elsevier Science Publishers (1989), Organic Reactions, Volumes 1-40, John Wiley and Sons (1991), March's Advanced Organic Chemistry, 4th Edition, John Wiley and Sons, and Larock's Comprehensive Organic Transformations, VCH Publishers Inc. (1989). Specifically, the compounds of this invention may be prepared by various methods known in the art of organic chemistry in general and nucleoside and nucleotide analogue synthesis in particular. General reviews of the preparation of nucleoside and nucleotide analogues include 1) Michelson, A. M., The Chemistry of Nucleosides and Nucleotides, Academic Press, New York (1963), 2) Goodman, L., Basic Principles in Nucleic Acid Chemistry, Vol. 1, Ch. 2, Academic Press, New York (1974), and 3) Zorbach, W. et al., eds., Synthetic Procedures in Nucleic Acid Chemistry, Vols. 1 and 2, Wiley, New York (1973).

Embodiments of the present invention will now be described by way of example only with respect to the following Examples.

### Preparation of Compounds

### Step 1

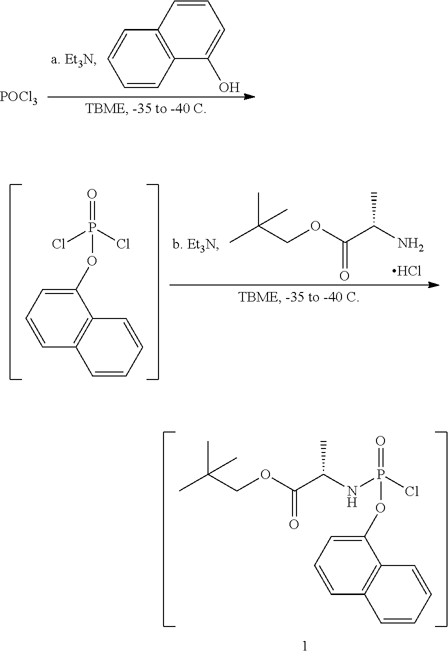

Charge MTBE (3.06 kg, ca. 4 L) to a 10 L CHEMGLASS® reactor flushed with nitrogen. The reactor was cooled with a chiller which was set at −40° C. POCl₃ (201.25 g, 1.3 mol) was added in one portion, followed by 1-naphthol (187.38 g, 1.3 mol). The crude was agitated for 5 minutes, and then triethylamine (131.52 g, 1.3 mol) was added over a course of 30 minutes, while maintaining internal temperature below −25° C. throughout the addition. The resulting white slurry was agitated for additional 30 minutes. To the reactor was charged with (S)-neopentyl 2-aminopropanoate hydrochloride salt (254.34 g, 1.3 mol) in one portion, followed by slow addition of triethylamine (263.04 g, 2.6 mol) over a course of 30 minutes. Agitation was resumed for an additional 2 hours before warming up to 0° C. The white slurry was filtered and rinsed with MTBE (2×100 mL). The filtrate was collected and used as is for next step without further purification (77% solution yield, 10.3 wt % in MTBE).

### Step 2: Preparation of Calcium Salt

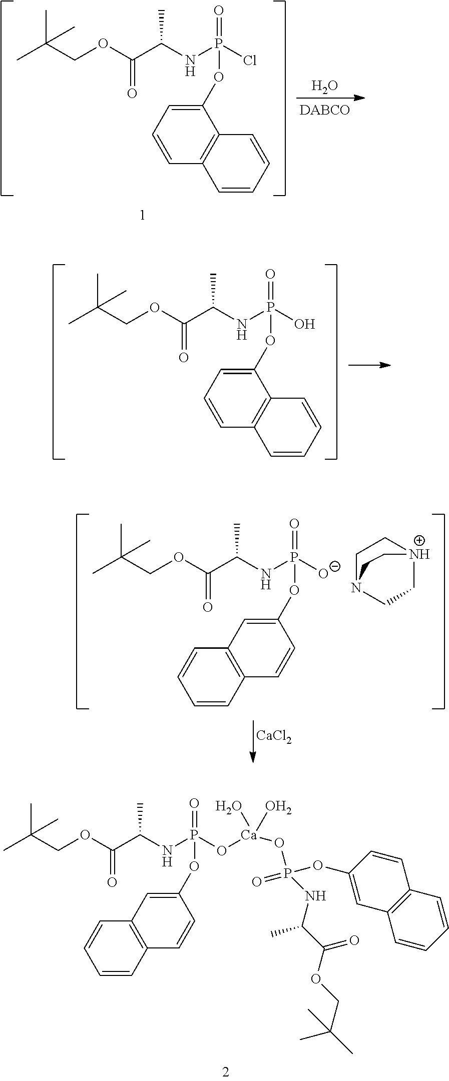

Charge phosphoric chloride as a solution in MTBE (1.49 kg, 10.3 wt %, 0.4 mol) to a 2 L CHEMGLASS® reactor. The reactor was cooled with a chiller which was set at 5° C. DABCO (134.83 g, 1.2 mol) was added in one portion. The crude was agitated for 30 minutes, and then water (72.18 g, 4.0 mol) was added over a course of 10 minutes, with internal temperature maintaining below 10° C. throughout the addition. The resulting crude was agitated for additional 3 hours, at which point the hydrolysis reached completion. Solvent swap by distillation into 2-propanol (500 mL). The resulting slurry was warmed up to 50° C., and water (750 mL) was charged in one portion. To the solution was charged with aq. solution of calcium chloride (22.23 g, 0.2 mol, as a solution in 250 mL water). Resume agitation for additional 30 minutes at 50° C. The crude was charged with seeds (0.2 wt %), cooled to 20° C. over 1 hour and held at this temperature over 12 hours. The white slurry was filtered, rinsed with IPA/water (20/80 vol %, 2×100 mL), and dried in vacuum oven at 50° C. The calcium salt was obtained as white crystalline solid with desired quality (93 g, 60%). ¹H NMR (500 MHz, DMSO-d₆, 23° C.) δ=0.78 (s, 18H), 1.20 (d, J=6.9 Hz, 6H), 3.49 (d, J=10.4 Hz, 2H), 3.62 (d, J=10.4 Hz, 2H), 3.68 (b, 2H), 3.94-4.00 (m, 2H), 7.32 (dd, J=7.3, 7.3 Hz, 2H), 7.38-7.46 (m, 6H), 7.61 (d, J=7.5 Hz, 2H), 7.77 (dd, J=1.5, 7.8 Hz, 2H), 8.16-8.18 (m, 2H); 31P-NMR (500 MHz, DMSO-d₆, 23° C.) δ=−1.89 (m).

### Alternate Step 2: Preparation of Quinine Salt

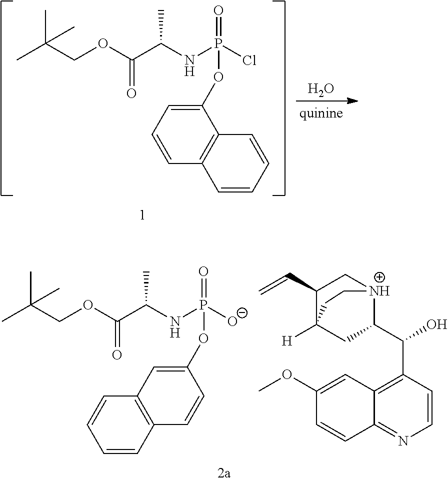

Charge phosphoric chloride as a solution in MTBE (745 g, 10.3 wt %, 0.2 mol) to a 2 L CHEMGLASS® reactor. The reactor was cooled with a chiller which was set at 5° C. tert-Amyl alcohol (500 mL) was charged, followed by quinine (194.58 g, 0.6 mol) in one portion. The crude was agitated for 30 minutes, and then water (36.02 g, 2.0 mol) was added over a course of 10 minutes, with internal temperature maintaining below 15° C. throughout the addition. The resulting crude was agitated for additional 18 hours, at which point the hydrolysis reached completion. MTBE was removed by distillation at 200 torr. Upon end of distillation, the resulting slurry was warmed up to 50° C., and methanol (250 mL) was charged to the crude, followed by water (300 mL). Agitation was resumed for additional 30 minutes at 50° C. and the crude was cooled to 20° C. over 1 hour and held at this temperature over 12 hours. The white slurry was filtered, rinsed with tert-amyl alcohol/MeOH/water (50/20/30 vol %, 2×100 mL), and dried in vacuum oven at 50° C. The quinine salt was obtained as white crystalline solid with desired quality (69 g, 51%). ¹H NMR (500 MHz, CD₂Cl₂, 23° C.) δ=0.85 (s, 9H), 1.26 (d, J=6.9 Hz, 3H), 1.21-1.27 (m, 1H), 1.64-1.71 (m, 1H), 1.99-2.07 (m, 5H), 2.53-2.58 (m, 1H), 2.80-2.85 (m, 1H), 2.94-3.00 (m, 1H), 3.16 (t, J=10.7 Hz, 1H), 3.29 (t, J=8.8 Hz, 1H), 3.55 (d, J=10.4 Hz, 1H), 3.71 (d, J=10.4 Hz, 1H), 3.81 (s, 3H), 4.04-4.10 (m, 1H), 4.23-4.30 (m, 1H), 4.95-5.00 (m, 2H), 5.50-5.57 (m, 1H), 6.34 (s, 1H), 7.25-7.30 (m, 3H), 7.36-7.43 (m, 2H), 7.48 (d, J=7.8 Hz, 1H), 7.54 (ddd, J=0.9, 1.2, 7.8 Hz, 1H), 7.62-7.63 (m, 1H), 7.75-7.77 (m, 1H), 7.95 (d, J=9.1 Hz, 1H), 8.23-8.25 (m, 1H), 8.61 (d, J=4.4 Hz, 1H); 31P-NMR (500 MHz, CD₂Cl₂, 23° C.) δ=+3.18 (s).

### Step 2b: Preparation of the DBU Salt

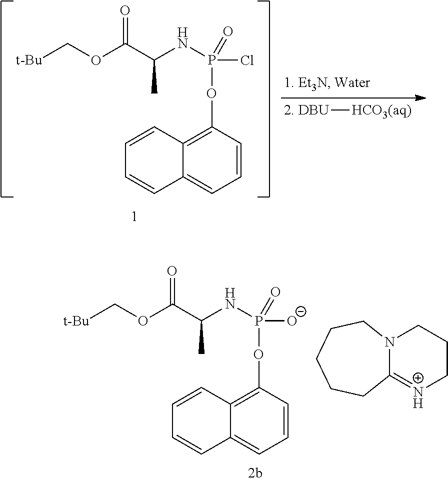

To a cooled solution (0° C.) of chlorophosphate in MTBE (0.23M, 230 mL, 52.4 mmol) was added with water (9.5 mL, 0.52 mol, 10 equiv) followed by triethylamine (75 mL, 0.52 mol, 10 equiv) in one portion. Stirred at ambient temperature for 3 h. To this solution was added DBU-carbonate (aq. 0.30M, 530 mL, 2 equiv). The layers were separated and the aqueous later was extracted with CH₂Cl₂ (2×250 mL). The solution was dried over MgSO₄. Filtered to provide the titled compound (21.7 g, 80% yield) as a 4.8 wt % solution in DCM. ¹H NMR (500 MHz, CDCl₃, 23° C.) δ=0.87 (s, 9H), 1.26 (t, J=7.4, Hz, 3H) 1.30 (d, J=6.9 Hz, 3H), 1.65-1.72 (m, 3H), 1.75-1.80 (m, 2H), 1.95-2.01 (m, 2H), 1.98-2.0 (m, 2H), 2.60-2.63 (m, 2H), 2.80-2.85 (m, 2H), 3.11 (q, J=7.3 Hz, 2H), 3.28 (t, J=5.8 Hz, 2H), 3.49 (t, J=5.8 Hz, 2H), 3.56-3.57 (m, 2H), 3.58 (d, J=10.4 Hz, 1H), 3.67 (d, J=10.4 Hz, 1H), 4.04 (dq, J=7.1 Hz, 1H), 7.35-7.40 (m, 1H), 7.45-7.50 (m, 2H), 7.53 (d, J=8.2 Hz, 1H), 7.57 (d, J=8.2, Hz, 1H), 7.8-7.83 (m, 1H), 8.29-8.31 (m, 1H); 31P-NMR (500 MHz, CDCl₃, 23° C.) δ=+1.65 (s).

### Step 3: Preparation of Phosphoramidate Side Chain

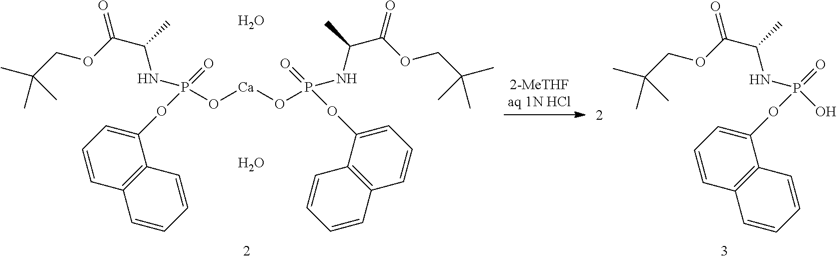

To a 30 mL sep funnel was added the calcium salt, 2 (1.00 g, 1.24 mmol) and 2-methyltetrahydrofuran (10.0 mL). Salts 2a and 2b may also be used. The suspension was washed with a 1 N aqueous solution of hydrochloric acid (2×10.0 mL), after which the clear homogeneous organic layer was washed with brine (2×10.0 mL), dried over sodium sulfate, filtered and concentrated in vacuo to afford a clear oil (630 mg, 69%). The oil was dissolved in THF (6.7 mL). PHENOMENEX® Kinetex C18 2.6 um 4.6×150 mm. A=0.05% TFA MeOH (20%)/water (80%); B=0.05% TFA MeOH (80%)/water (20%). Gradient: A=100%; t=5 B=30; t=25 B=50; t=30 B=100. Flow rate=1 mL/min; Compound 3 rt=22.13 min.

### Step 4

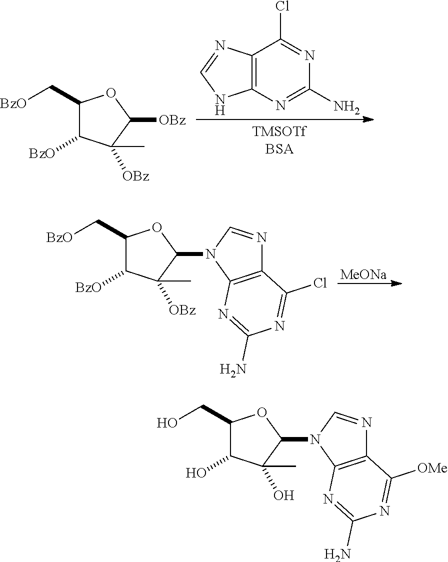

The chloro-nucleoside can be prepared readily from the tetrabenzoate and a chloro-purine derivative, as shown above. This chemistry is well described in the literature (WO 2004/003138; J. Med. Chem., 47:2283 (2004); WO 2006/122207; Bioorg. Med. Chem. Lett., 17:2456 (2007); WO 2010/081082; Bioorg. Med. Chem. Lett., 20:4850 (2010); Bioorg. Med. Chem. Lett., 21:6007 (2011); WO 2011/123586; Bioorg. Med. Chem. Lett., 21:6788 (2011); WO 2012/048013). Methoxylation of the chloro-purine derivative with sodium methoxide has also been described in detail (Bioorg. Med. Chem. Lett., 21, 6007 (2011); WO 2011/123586; Bioorg. Med. Chem. Lett., 20:4850 (2010); WO 2010/081082), the disclosures of which are herein incorporate by reference.

### Step 5

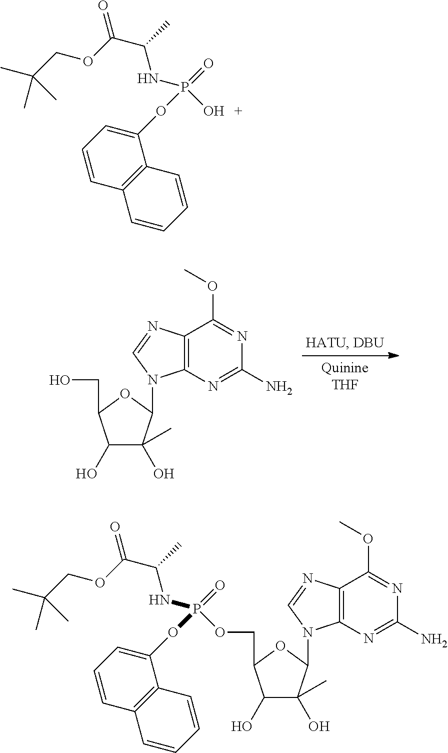

A solution of the phosphoric acid (6.4 mmol) in 2MeTHF (13 mL) was treated with Hunig's base (560 μL) and HATU (2.4 g). The nucleoside (1 g) and quinine (938 mg) were then added and the mixture heated to 50° C. for 4 h. In-process analysis indicated product in 90% yield as a 6.7:1 diastereomeric mixture favoring P(S). PHENOMENEX® Kinetex C18 2.6 nm 4.6×150 mm. A=0.05% TFA MeOH (20%)/water (80%); B=0.05% TFA MeOH (80%)/water (20%). Gradient: A=100%; t=5 B=30; t=25 B=50; t=30 B=100. Flow rate=1 mL/min; P(R) isomer rt=23.59 min; P(S) isomer rt=24.71 min.

What is claimed is:

1. A method for preparing a compound of formula I having the following structure, or a pharmaceutically acceptable salt thereof: 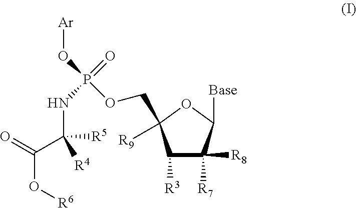
wherein
Base is a naturally occurring or modified purine or pyrimidine base linked to the furanose ring through a carbon or nitrogen atom;
Ar is selected from phenyl, naphthyl, 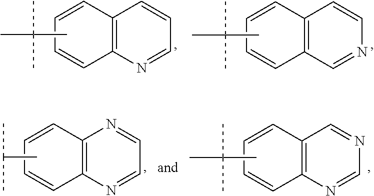
any of which are optionally substituted with C₁-C₆alkyl, C₁-C₆alkoxy, di(C₁-C₆)alkylamino or C₁-C₆alkylcarboxy(C₁-C₆)alkyl-;
R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;
R⁴ and R⁵ are independently selected from hydrogen, C₁-C₆alkyl optionally substituted with alkylthio, benzyl optionally substituted with one or more halo, C₁-C₆alkyl, or C₁-C₆alkoxy, phenyl optionally substituted with one or more halo, C₁-C₆alkyl, or C₁-C₆alkoxy;
R⁶ is selected from C₁-C₁₀alkyl, C₃-C₈cycloalkyl, C₃-C₈cycloalkyl-alkyl-, phenyl(C₁-C₆)alkyl- optionally substituted with C₁-C₆alkyl, C₁-C₆alkoxy, and halo, indanyl and heterocycloalkyl;
R⁷ is selected from the group consisting of H, alkyl, —OH, OP, wherein P is a protecting group, OCH₃, halo, NH₂;
R⁸ is selected from the group consisting of H, CH₃, CH₂F, CHF₂, CF₃, F, CN, —OH, —OP wherein P is a protecting group, halo, alkyl, alkenyl, and alkynyl; and
R⁹ is H, C₁₋₄ alkyl, CN, halo, —OH, —CH₂CN, —CH₂NH₂, vinyl, C₂-C₄ alkynyl, O—C₁₋₆ alkyl, —CH₂F, N₃,
in the presence of an activator, a base, and optionally an additive;
comprising contacting a compound having the following Formula II, or a salt thereof: 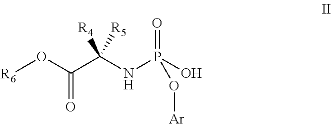
with a nucleoside compound of Formula III: 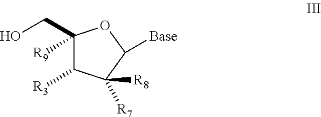
wherein
R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;
R⁷ is selected from the group consisting of H, alkyl, —OH, OP, wherein P is a protecting group, OCH₃, halo, N₃, NH₂;
R⁸ is selected from the group consisting of H, CH₃, CH₂F, CHF₂, CF₃, F, CN, —OH, —OP wherein P is a protecting group, halo, alkyl, alkenyl, and alkynyl; and
R⁹ is H, C₁₋₄ alkyl, CN, halo, —OH, —CH₂CN, —CH₂NH₂, vinyl, C₂-C₄ alkynyl, O—C₁₋₆ alkyl, —CH₂F, N₃,
in the presence of an activator, a base, and optionally an additive.
comprising contacting a compound having the following Formula II, or a salt thereof: 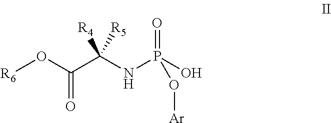
with a nucleoside compound of Formula III: 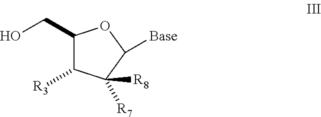
wherein
R³ is OH, H, alkyl, C₂-C₄ alkenyl, C₂-C₄ alkynyl, vinyl, N₃, CN, Cl, Br, F, I, NO₂, OC(O)O—C₁₋₄ alkyl, —OC₁₋₁₀ alkyl, haloalkyl or —OH;
R⁷ is selected from the group consisting of H, alkyl, —OH, OP, wherein P is a protecting group, OCH₃, halo, NH₂; and
R⁸ is selected from the group consisting of H, CH₃, CH₂F, CHF₂, CF₃, F, CN, —OH, —OP wherein P is a protecting group, halo, alkyl, alkenyl, and alkynyl;
in the presence of an activator, a base, and optionally an additive.

2. The process according to claim 1 wherein the activator is a uronium or phosphonium activator.

3. The process according to claim 1 wherein the base is an organic base.

4. The process according to claim 3 wherein said organic base is triethylamine, Hunig's base, DMAP, DBU or 1,8-diazabicyclo[5.4.0]undec-7-ene.

5. The process of claim 1 wherein said additive is quinine or a quinine derivative.

6. The process of claim 1 wherein Ar is naphthyl or phenyl R³ is —OH; R⁴ and R⁵ are H or lower alkyl, independently, and R⁹ is H.

7. The process of claim 1 wherein R⁸ is -alkyl or halo.

8. The process of claim 1 wherein R⁶ is selected from H, optionally substituted C₁-C₁₀ alkyl, wherein said substituent is selected from halo, methoxy, alkylamino, benzyl, and allyl which may be optionally substituted with halo, methoxy, benzyl, alkylamino, trialkylsilyl; optionally substituted phenyl (C₁-C₆) alkyl, wherein said substituents are independently selected from halo, methoxy, benzyl, alkylamino, and allyl which may be optionally substituted with halo, methoxy, benzyl, alkylamino, and trialkylsilyl.

9. A process for preparing a compound having the following Formula IV: 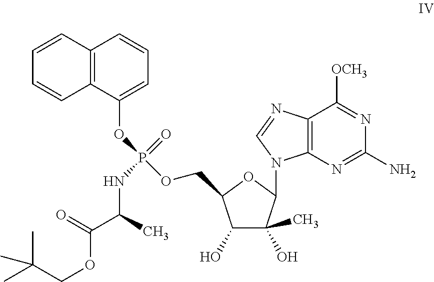 [SMILES: COc1nc(N)nc2c1ncn2C1O[C@H](CO[P@@](=O)(N[C@@H](C)C(=O)OCC(C)(C)C)Oc2cccc3ccccc23)[C@@H](O)[C@@]1(C)O]
comprising contacting a compound having Formula V: 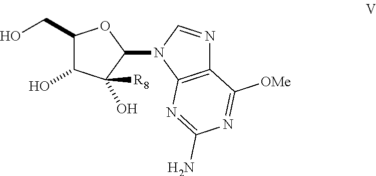
with a compound having Formula VI, or a salt thereof: 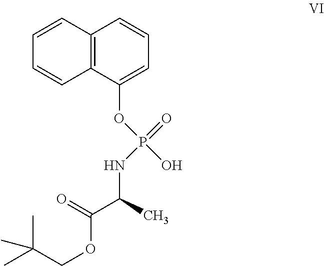 [SMILES: C[C@H](NP(=O)(O)Oc1cccc2ccccc12)C(=O)OCC(C)(C)C]
in the presence of an activator, a base and optionally an additive.

10. The process of claim 9 wherein said activator is selected from a phosphonium or a uronium activator, said base is Hunig's base and said addititive is quinine or a quinine derivative.

11. A process for making a compound having the following Formula X: 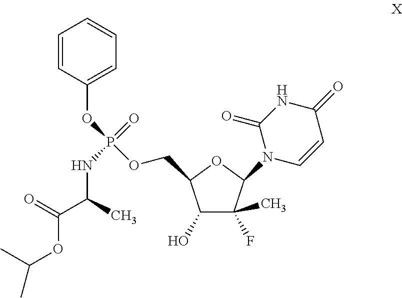 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
comprising contacting a compound having the Formula XI: 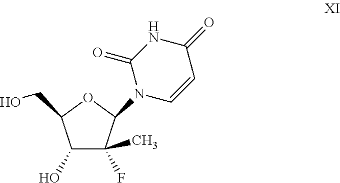 [SMILES: C[C@@]1(F)[C@H](O)[C@@H](CO)O[C@H]1n1ccc(=O)[nH]c1=O]
with a compound having the Formula XII 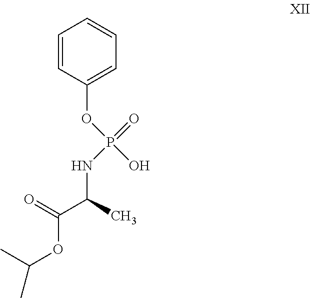 [SMILES: CC(C)OC(=O)[C@H](C)NP(=O)(O)Oc1ccccc1]
or a salt thereof in the presence of an activator, a base, and an optional additive.

12. A compound having the following Formula II, or a salt or hydrate thereof: 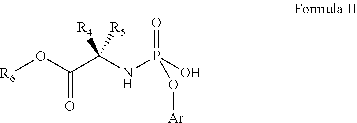
wherein
R⁴ and R⁵ are independently selected from hydrogen, C₁-C₆alkyl optionally substituted with alkylthio, benzyl optionally substituted with one or more halo, C₁-C₆alkyl, or C₁-C₆alkoxy, phenyl optionally substituted with one or more halo, C₁-C₆alkyl, or C₁-C₆alkoxy;
R⁶ is selected from C₁-C₁₀alkyl, C₃-C₈cycloalkyl, C₃-C₈cycloalkyl-alkyl-, phenyl(C₁-C₆)alkyl- optionally substituted with C₁-C₆alkyl, C₁-C₆alkoxy, and halo, indanyl and heterocycloalkyl; and
Ar is selected from phenyl, naphthyl, 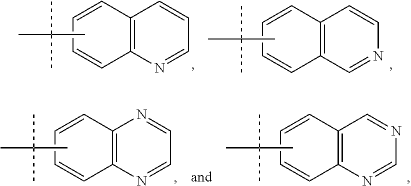
any of which are optionally substituted with C₁-C₆alkyl, C₁-C₆alkoxy, di(C₁-C₆)alkylamino or C₁-C₆alkylcarboxy(C₁-C₆)alkyl.

13. A compound of claim 12 having the following structure: 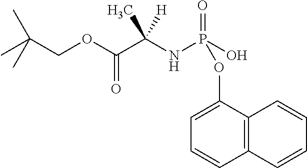 [SMILES: C[C@@H](NP(=O)(O)Oc1cccc2ccccc12)C(=O)OCC(C)(C)C]
or a salt, hydrate or solvate thereof.

14. The compound of claim 13 wherein said salt is:

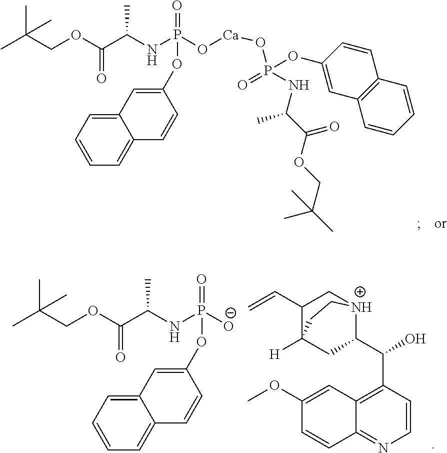

15. A compound having the following Formula XII: 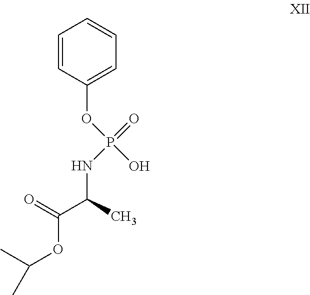 [SMILES: CC(C)OC(=O)[C@H](C)NP(=O)(O)Oc1ccccc1]
or a salt or hydrate thereof.
